(19) United States
(12) Patent Application Publication
(10) Pub. No.: US 2024/0285748 A1
(43) Pub. Date: Aug. 29, 2024

(54) VETERINARY VIRAL VECTOR

(71) Applicant: VAXXINOVA INTERNATIONAL B.V., Gelderland (NL)

(72) Inventors: Vikram Verma, New London, MN (US); Darren E. Straub, New London, MN (US); Daryll A. Emery, Wilmar, MN (US)

(21) Appl. No.: 18/572,736

(22) PCT Filed: Jun. 21, 2022
(86) PCT No.: PCT/IB2022/000378

§ 371 Date: Dec. 20, 2023

Related U.S. Application Data
(60) Provisional application No. 63/212,998, filed on Jun. 21, 2021.

(51) Int. Cl. A61K 39/12; C07K 14/005; C12N 7/00

(52) U.S. Cl. CPC A61K 39/12; C07K 14/005; C12N 7/00; C12N 2770/00021; C12N 2770/00022; C12N 2770/00034

(57) ABSTRACT

Provided herein are veterinary viral vectors and vaccines based on genetically engineered Pichinde viruses that include three or more genomic segments. The first genomic segment includes a coding region encoding a Z protein and a coding region encoding a L RdRp protein. The second genomic segment includes a coding region encoding a nucleoprotein (NP) and the third genomic segment includes a coding region encoding a glycoprotein. At least one of the second and third genomic segments further includes a donor gene sequence encoding an adenovirus capsid protein in full length or any functional fragment thereof. Further provided are methods for using a reverse genetics system, and methods for producing an immune response against adenovirus or Hemorrhagic Enteritis Virus (HEV) infection of a subject such as a turkey.

### CROSS-REFERENCE TO RELATED APPLICATIONS

This application is being filed on Jun. 21, 2022, as a PCT International Patent application and claims the benefit of and priority to U.S. Provisional Patent Application No. 63/212,998, filed Jun. 21, 2021, the entire disclosure of which is incorporated by reference in its entirety.

### SEQUENCE LISTING SUBMISSION

The present application includes a Sequence Listing in electronic format as a txt file titled “14579.0002USP1-SEQUENCE-LISTING,” which was submitted along with the present application. The contents of txt file “14579.0002USP1-SEQUENCE-LISTING” are incorporated by reference herein.

### BACKGROUND

One of the principal diseases which suppresses the immune system in birds is caused by infection with Hemorrhagic Enteritis Virus (HEV). HEV belongs to the Adenoviridae family. This family consists of serotypes that infect mammals (Mastadenoviridae) and avian (Aviadenoviridae) [Shenk T. Virology, pp. 2111-2148 (1996), B. N. Fields, D. M. Knipe and P. M. Howley (Eds) Lippincott-Raven New York]. HEV, together with marble spleen disease of pheasant and splenomegaly virus of chickens and turkey's, are classified as type II avian adenovirus (Ad) [Domermuth C. H. and Gross W. B., Diseases of Poultry 8th ed. Pp.511-516, H. J. Barnes, B. W. Calnck, W. B. Reid and Yoder H. W. (Eds) Iowa State University Press (1984)]. Other serologically distinct adenoviruses include type I Ad and type III Ad, isolated from chickens infected by fowl Ad (FAV)1-12 and egg drop syndrome (EDS) virus, respectively.

The HEV virus infects birds and causes a disease which is characterized by depression, splenomegaly, intestinal hemorrhages and immuno-suppression [Domermuth C. H., & Gross W. B. Diseases of Poultry, 9th Edition, Hofstad M. S. et al. Eds. Iowa State University Press, Ames, Iowa (1991)]. The virus replicates in B cells and macrophages [Suresh M. et al., Pathogenesis of type II avian adenovirus infection in turkeys: in vivo immune cell tropism and tissue distribution of the virus, J. Virol. 70:30-36 (1996)] and is concentrated in large amounts in the spleen. Since B cells play an important role in the primary immune response of the bird, viral infection of this cell lineage results in immunosuppression which in turn affects the ability of the bird to mount an effective immune response against secondary bacterial infection such as E. coli, often resulting in high mortality and economic loses.

Infection of birds by the HEV is especially prevalent during the ages of 7 to 9 weeks [Domermuth C. H., et al., Diseases of Poultry, (1984), supra]. Younger birds 3-4 weeks of age are considered resistant to infection, primarily due to the presence of maternal antibodies that may last up to 4-6 weeks of age [Van den Hurk, J. V., Quantitation of hemorrhagic enteritis virus antigen and antibody using enzyme-linked immunosorbent assays, Avian Dis. 30:662-671 (1986): Harris J. R. et al., Hemorrhagic Enteritis in Two-and-One-Half-Week-Old Turkey Poults, Avian Dis. 21:120-122 (1977): Fadly, A. M. et al., Hemorrhagic Enteritis of Turkey's: Influence of Maternal Antibody and Age at Exposure, Avian Dis. 33:778-786 (1989)]. The rate of mortality of infected birds is high and, since the immune response is damaged, the surviving birds exhibit high vulnerability to other viral and/or bacterial diseases. Moreover, infection with HEV reduces the effectiveness of response to various vaccines. As a result of lowered resistance, an outbreak of a HEV infection may further lead to outbreaks of other diseases (i.e., secondary infection). Naturally, such events result in heavy financial loss to the breeders. Infectious diseases in animals, and in particular in farm animals, are one of the most important economic factors in agriculture. The minimalization of losses from diseases, by means of effective vaccines, plays a major part in achieving profit in today's intensive agricultural industry. The health of domesticated animals depends on management, on a proper vaccination system and on the availability of effective vaccines.

HE is an economically important viral disease in commercial turkey production especially in the USA and Canada. Financial losses of the disease were primarily due to the immunosuppressive effects of the virus which resulted in secondary bacterial infections in susceptible birds. However, due to the extensive use of live HE vaccines and following strict biosecurity on commercial facilities, highly pathogenic outbreaks of HE, are now rare in commercial operations. Nevertheless, replicative competent live HE vaccines currently used to control HE still induce immunosuppression following the acute stage of viral replication of the live vaccine. The immune system becomes depressed after vaccination of the live vaccine due to its ability to replicate in B cells during HEV infection and resulting in macrophage depletion (Sponenberg 1985). This immunosuppressive effect can interfere in vaccination protocols as well as predispose birds to opportunistic bacterial pathogens, such as E. coli (Sharma, 1991).

It is therefore highly desirable for a safe and specific vaccine for efficient prevention of infection with adenovirus or HEV in birds, especially in poultry. It is also desirable that the vaccine be easy and cost-effective to produce and be suitable for administration on an industrial basis. It is further desirable that successful immunization with the vaccine be easily detectable and confirmable.

### SUMMARY

The present application provides compositions and methods regarding veterinary viral vectors and vaccines against infection with adenovirus or HEV, based on genetically engineered Pichinde viruses and reverse genetics systems for making the same.

In one embodiment, a genetically engineered Pichinde virus includes three genomic segments. The first genomic segment includes a coding region encoding a Z protein and a coding region encoding a L RNA-dependent RNA polymerase (L RdRp) protein. The second genomic segment includes a coding region encoding a nucleoprotein (NP) and a first restriction enzyme site. The third genomic segment includes a coding region encoding a glycoprotein (GPC) and a second restriction enzyme site. At least one of the second and the third genomic segment further comprises a donor gene sequence encoding an adenovirus capsid protein or a functional fragment thereof. In one embodiment, the adenovirus capsid protein is a hemorrhagic enteritis virus (HEV) capsid protein. In one embodiment, the HEV capsid protein is a HEV full length fiber protein (HEVFP). In one preferred embodiments, the HEV capsid protein is HEVFP that is codon optimized to avian species.

The Pichinde virus according to the present disclosure comprises one or more nucleic acid sequences at least 80% identical to any one of sequences set forth in SEQ ID NOs: 5-6 and 8-20.

In one embodiment, the donor gene sequence comprises a nucleic acid sequence encoding the hemorrhagic enteritis virus (HEV) capsid protein or a functional fragment thereof. In certain embodiments, the donor gene sequence comprises a nucleic acid sequence encoding a protein, wherein the protein has an amino acid sequence at least 80% identical to any one of SEQ ID NOs: 7, and 21-22. In certain embodiments, the donor gene sequence has at least 80% identity to any of SEQ ID NOs: 15, and 18-20.

In one embodiment, the second genomic segment includes a multiple cloning site, and the first restriction enzyme site is part of the multiple cloning site. The donor gene sequence of the second genomic segment may be inserted at the first restriction site. In one embodiment, the third genomic segment includes a multiple cloning site, and the second restriction enzyme site is part of the multiple cloning site. In one embodiment, the donor gene sequence of the third genomic segment may be inserted at the second restriction site. In one embodiment, the second genomic segment includes a first donor gene sequence encoding a first adenovirus capsid protein inserted at the first restriction site, and the third genomic segment includes a second donor gene sequence encoding a second adenovirus capsid protein inserted at the second restriction site. In one embodiment, at least one of the first, the second, and the third genomic segments further includes a reporter gene encoding a detectable marker. The first donor gene sequence and the second donor gene sequence may be the same or may be different. The first adenovirus capsid protein and the second adenovirus capsid protein may be the same or may be different. In one embodiment, both the first and the second donor gene sequences each comprise a nucleic acid sequence having at least 80% identity to any of SEQ ID NOS: 15 and 18-20.

In one embodiment, each of the genomic segments further comprises a regulatory sequence including but not limited to a promotor, a transcription initiation start site, a Kozak consensus sequence, a ribosome binding site, an RNA processing signal, a transcription termination site, a polyadenylation signal, or a combination thereof.

In one embodiment, the present genetically engineered Pichinde virus further comprises a fourth genomic segment encoding a T7 RNA polymerase.

Also provided herein is a collection of vectors. In one embodiment, the vectors are plasmids. In one embodiment, the collection includes a first vector encoding a first genomic segment including a coding region encoding a Z protein and a coding region encoding a L RdRp protein, wherein the first genomic segment is antigenomic, a second vector encoding a second genomic segment including a coding region encoding a nucleoprotein (NP) and a first restriction enzyme site, wherein the second genomic segment is antigenomic, and a third vector encoding a third genomic segment includes a coding region encoding a glycoprotein and a second restriction enzyme site, wherein the third genomic segment is antigenomic, and wherein at least one of the second and the third genomic segment further comprises a donor gene sequence, wherein the donor gene sequence encodes an adenovirus capsid protein or a fragment thereof. In one embodiment, the adenovirus capsid protein is a hemorrhagic enteritis virus (HEV) capsid protein. In one embodiment, the HEV capsid protein is a HEV fiber protein (HEVFP). In one embodiment, the HEV capsid protein is a full length HEVFP. In one embodiment, the donor gene sequence comprises a nucleic acid sequence having at least 80% identity to SEQ ID NO: 15. In one embodiment, the HEV capsid protein comprises a knob protein, a shaft protein, and optionally an adjacent part of the shaft protein. In one embodiment, the capsid protein comprises a hexon protein. In some embodiments, the donor gene sequence comprises a nucleic acid sequence having at least 80% identity to SEQ ID NOs: 15 and 18-20.

In one embodiment, the second genomic segment includes a multiple cloning site, and the first restriction enzyme site is part of the multiple cloning site. The donor gene sequence of the second genomic segment may be inserted at the first restriction site. In one embodiment, the third genomic segment includes a multiple cloning site, and the second restriction enzyme site is part of the multiple cloning site. In one embodiment, the donor gene sequence of the third genomic segment may be inserted at the second restriction site. In one embodiment, the second genomic segment includes a first donor gene sequence encoding a first adenovirus capsid protein inserted at the first restriction site, and the third genomic segment includes a second donor gene sequence encoding a second adenovirus capsid protein inserted at the second restriction site. In one embodiment, at least one of the first, the second, and the third genomic segments further includes a reporter gene encoding a detectable marker. The first donor gene sequence and the second donor gene sequence may be the same or may be different. The first adenovirus capsid protein and the second adenovirus capsid protein may be the same or may be different. In one embodiment, both the first and the second donor gene sequences each comprise a nucleic acid sequence having at least 80% identity to any one of SEQ ID NOs: 15 and 18-20.

In one embodiment, each of the genomic segments further comprises a regulatory sequence including but not limited to a promotor, a transcription initiation start site, a Kozak consensus sequence, a ribosome binding site, an RNA processing signal, a transcription termination site, a polyadenylation signal, or a combination thereof.

In one embodiment, the present collection of vectors further comprises a fourth vector encoding a fourth genomic segment, wherein, the fourth genomic segment encodes a T7 RNA polymerase.

Further provided are methods. In one embodiment, a method includes making a genetically engineered Pichinde virus as described herein. The method includes introducing into a cell the collection of vectors described herein and incubating the cells in a medium under conditions suitable for expression and packaging of the first, second, third, and/or the fourth genomic segment into a virus particle. The method may also include isolating an infectious virus particle from the medium.

Also provided herein is a reverse genetics system for making a genetically engineered Pichinde virus, wherein the system includes three vectors. The first vector encodes a first genomic segment including a coding region encoding a Z protein and a coding region encoding a L RdRp protein, wherein the first genomic segment is antigenomic, the second vector encodes a second genomic segment including a coding region encoding a nucleoprotein (NP) and a first restriction enzyme site, wherein the second genomic segment is antigenomic, and the third vector encodes a third genomic segment includes a coding region encoding a glycoprotein and a second restriction enzyme site, wherein the third genomic segment is antigenomic, and wherein at least one of the second and the third genomic segment further comprises a donor gene sequence encoding an adenovirus capsid protein or a fragment thereof. In one embodiment, the adenovirus capsid protein is a hemorrhagic enteritis virus (HEV) capsid protein. In one embodiment, the donor gene sequence comprises a nucleic acid sequence having at least 80% identity to any one of SEQ ID NOs: 15 and 18-20. In one embodiment, the present reverse genetics system further comprises a fourth vector encoding a fourth genomic segment, wherein the fourth genomic segment encodes a T7 polymerase.

In one embodiment, a method includes using a reverse genetics system, including introducing into a cell the vectors of genomic segments described herein, incubating the cell under conditions suitable for the transcription of the genomic segments and expression of the coding regions of each genomic segment. In one embodiment, the method also includes isolating infectious virus particles produced by the cell, wherein each infectious virus particle includes the three genomic segments. In one embodiment, the introducing includes transfecting a cell with the genomic segments. In one embodiment, the introducing includes contacting the cell with an infectious virus particle including the genomic segments. In one embodiment, the cell is ex vivo, such as a vertebrate cell. In some embodiments, the vertebrate cell is a mammalian cell, or a human cell, or an avian cell.

In one embodiment, a method includes producing an immune response in a subject. The method includes administering to a subject an infectious virus particle described herein. In one embodiment, the subject is a turkey. The immune response may include a humoral immune response, a cell-mediated immune response, or a combination thereof. The subject may have been exposed to or is at risk of exposure to adenovirus or HEV infection.

Also provided herein is an infectious virus particle as described herein, and a composition that includes an infectious virus particle described herein.

Also provided here are compositions or vaccines for vaccination of a turkey against Hemorrhagic Enteritis Virus (HEV). In one embodiment, a composition or vaccine comprises an engineered Pichinde viral vector according to the present disclosure, wherein the Pichinde viral vector comprises at least one donor gene sequence encoding a full-length HEV capsid protein. In one particular embodiment, the donor gene sequence is a codon-optimized sequence having at least 60% identity to SEQ ID NO: 20. In one embodiment, the full-length HEV capsid protein has an amino acid sequence having at least 60% identity to SEQ ID NO:7. The composition or vaccine provided herein may further comprise a pharmaceutically or veterinary acceptable carrier, excipient, vehicle, adjuvant, or combinations thereof.

In one embodiment, a method of vaccinating a turkey against Hemorrhagic Enteritis Virus (HEV) comprises at least one administration of the composition or vaccine according to the present disclosure. In some embodiments, the turkey is at least 1 day of age, or at least 2 days of age, or at least 3 days of age, or at least 4 days of age, or at least 5 days of age, or at least 6 days of age, or at least 7 days of age, or at least 1 week of age, or at least 2 weeks of age, or at least 3 weeks of age, or at least 4 weeks of age, or at least 5 weeks of age. In one embodiment, the method comprises a prime-boost administration regime. The prime-boost administration regime may comprise at least one primary administration and at least one booster administration of the composition or vaccine.

### BRIEF DESCRIPTION OF THE FIGURES

FIG. 1 shows schematic diagrams depicting the genome organization of the wild-type (WT) PICV genome (bi-segmented) and the tri-segmented rP18tri vaccine vector. FIG. 1(A) shows WT PICV genome which consists of two genomic RNA segments (L and S), each encoding two viral genes in opposite orientation. The L segment encodes the matrix protein Z and L polymerase protein, the S segment encodes the glycoprotein GPC and the nucleoprotein NP. IGR, intergenic region. FIG. 1(B) shows tri-segmented rP18tri vector system comprising three RNA segments, L, S1, and S2. S1 encodes GPC and contain one Multiple cloning site (MCS). S2 encodes NP and contain another MCS.

FIG. 2 shows vector maps of plasmids encoding S1 and S2 RNA segments. Plasmids rP18triS1 shown in FIG. 2(A) and rP 18triS2 shown in FIG. 2(B) are based on the PICV P18 reverse genetics system plasmid pP18S, which encodes the full-length P18 S RNA segment, followed by hepatitis delta virus ribozyme sequence (HDVRibo), under a T7 promoter.

FIG. 3 is a schematic illustration of the cloning location of the gene sequence encoding the HEV full-length fiber protein, or a codon-optimized HEV full-length HEV fiber protein, or various fragments of the HEV fiber protein in the PICV P18tri vector. The gene sequence encoding the HEV fiber protein or a fragment thereof was inserted in the Multiple Cloning Sites (MCS) within both the S1 and S2 plasmids using Nhe I and Kpn I restriction enzymes. FIG. 3(A) illustrates PICV-HEV-fiber-Knob-Shaft (KS): FIG. 3(B) illustrates PICV-HEV-fiber-Full Length (FL): FIG. 3(C) illustrates PICV-HEV-fiber-Full Length-codon-optimized (cFL): FIG. 3(D) illustrates PICV-HEV-Fiber-Hexon: FIG. 3(E) illustrates PICV-HEV-Hexon.

FIG. 4 is a schematic illustration of the production of the genetically modified Pichinde virus comprising the donor gene sequence encoding the HEV fiber protein of a fragment thereof. The PICV-HEVFP viral vaccine was generated by simultaneously transfecting three plasmids encoding the L, S1, and S2 RNA segments and another plasmid encoding T7 RNA polymerase into Bhk-21 cells. Supernatants containing the rescued PICV-HEVFP virus were collected from the transfected cells at 48-72 hr time points.

FIG. 5 illustrates the restriction digestion pattern from the PICV-HEVFP virus. From left to right: Lane 1 represents 100-1,500 bp ladder: lane 2 represents fragment containing the donor gene sequence encoding the HEV Fiber Protein, Lane 3 represents fragment without donor gene sequence encoding the HEV Fiber Protein.

FIG. 6 illustrates the comparative spleen/Body weight ratio of vectors rP18tri-HEV-hexon, rP18tri-HEV-fiber-(FL) and rP18tri-HEV-hexon/fiber after HEV challenge. It is noted that the protective efficacy of vector rP18tri-HEV-fiber-(FL) is compared to the efficacy of vectors rP18tri-HEV-hexon, rP18tri-HEV-hexon/fiber, as well as the commercial vaccine product Oralvax. The vector rP18tri-HEV-fiber-(FL) comprising the gene sequences coding for the HEV full length Fiber Protein reduced the incidence of splenomegaly compared to all test groups with a mean Spleen/Body weight ratio of 1.08.

FIG. 7 illustrates the comparative Spleen/Body weight ratio of vaccinates to control and Oralvax. The Vector (rP18tri-HEV-fiber-cFL) comprises the codon optimized sequence of the full length (cFL) fiber protein. It is noted that the protective efficacy of vector rP18tri-HEV-fiber-(cFL) is compared to the control and the commercial vaccine product, Oralvax. The codon optimized vector reduced the incidence of splenomegaly at each dose given compared to the vector control and Oralvax with the lowest effective dose of 1.0×10⁵ PFU showing a mean Spleen/Body weight ratio of 1.04.

FIG. 8 shows the comparative spleen/body weight ratio of vaccinates to control and Oralvax, according to Example 14. The vector (rP18tri-HEV-fiber-FL) utilized the non-codon optimized sequence of the full length fiber protein.

### DETAILED DESCRIPTION

The present application provides compositions and methods regarding veterinary viral vectors and vaccines based on genetically engineered Pichinde (PICV) virus. The veterinary viral vectors and vaccines include a donor gene sequence inserted into one or more genomic segments of a genetically engineered Pichinde virus. The inserted donor gene sequence can express adenovirus capsid protein antigen to induce strong humoral and cell-mediated immunity against adenovirus or Hemorrhagic Enteritis Virus (HEV). Additionally, this can be accomplished with little anti-vector immunity in vivo, a high level of biosafety, and no adverse effects.

The genetically modified Pichinde virus-based reverse genetics system described herein has multiple advantages. Pichinde virus is of high biosafety. The wild type Pichinde virus has not been shown to produce disease in humans or animals [Buchmeier M, et al., Serological Evidence of Infection by Pichinde Virus Among Laboratory Workers. Infect Immun. 9(5): 821-823, (1974): Walker C. M., et al., Generation of memory cell-mediated immune responses after secondary infection of mice with pichinde virus. J Immunol. 132(1):469-74, (1984)]. Additionally, there is evidence that Pichinde virus can cause asymptomatic human infections in a laboratory setting. For instance, 46% of laboratory personnel working with the virus are serum positive but do not show a distinct illness [Buchmeier, Arenaviridae: the viruses and their replication. In: Knipe and Howley (eds), Fields Virology. 5th ed. Philadelphia, PA: Lippincott Williams & Wilkins. pp. 1791-1827]. The modified Pichinde virus described herein is further attenuated, compared to the parental virus used in the human-infection study reported by Buchmeier et al. (supra). The modified Pichinde virus is genetically stable through serial passages in cell cultures. General human populations are not known to have prior exposure to Pichinde virus, which makes it an ideal vector for vaccine development due to the lack of pre-existing immunity against this Pichinde virus vector.

As used herein, “genetically modified” and “genetically engineered” refer to a Pichinde virus which has been modified and is not found in any natural setting. For example, a genetically modified Pichinde virus is one into which has been introduced an exogenous polynucleotide, such as a restriction endonuclease site. Another example of a genetically modified Pichinde virus is one which has been modified to include three or more genomic segments.

Pichinde virus has been previously used as a model virus to study viral hemorrhagic fever infection. A reverse genetics system was previously developed to generate infectious PICV viruses from two plasmids that encode the viral large (L) and small (S) RNA segments [Lan et al., Development of Infectious Clones for Virulent and Avirulent Pichinde Viruses: a Model Virus To Study Arenavirus-Induced Hemorrhagic Fevers, J Virol 83:6357-6362 (2009)]. In a separate study, an advanced reverse genetics system was created to produce recombinant infectious PICV viruses from 3 separate plasmids that are referred to as the tri-segmented PICV system. [Dhanwani R., et. al, A novel live Pichinde virus-based vaccine vector induces enhanced humoral and cellular immunity upon a booster dose. J Virol 90(5):2551-2560, (2015)]. These recombinant viruses carry 3 viral genomic RNA segments that encode for all of the viral gene products as well as two foreign genes. This tri-segmented PICV system could be used as a novel vaccine vector to deliver the hemagglutination (HA) and the nucleo-protein (NP) of the influenza virus A/PRS strain. Mice immunized with these recombinant viruses are protected against lethal influenza virus challenge as evidenced by the survival of the animals afforded by the high levels of HA neutralizing antibodies and NP-specific cytotoxic T lympho-cyte (CTL) responses to viral infection. These tri-segmented recombinant PICV viruses do not induce strong anti-PICV vector immunity, thus making them ideal candidates in a prime-boost vaccination strategy in order to induce cross-reactive immunity. [Dhanwani R., et. al. J Virol (2015), supra]. Examples of a genetically modified tri-segmented Pichide virus could also be found in U.S. Pat. No. 10,533,159, the disclosure of which is hereby incorporated by reference in its entirety.

Adenoviruses (the family of Adenoviridae) are medium-sized, nonenveloped (without an outer lipid bilayer) viruses with an icosahedral nucleocapsid containing a double stranded DNA genome. Adenovirus family consists of serotypes that infect mammals (Mastadenoviridae) and avian (Aviadenoviridae) [Shenk T. Virology, pp. 2111-2148 (1996), B. N. Fields, D. M. Knipe and P. M. Howley (Eds) Lippincott-Raven New York.] Adenoviruses normally have a capsid consisting of three main exposed structural proteins: hexon, fiber, and penton base. These three proteins are referred to herein as capsid proteins. Hexon accounts for the majority of the structural components of the capsid, which consists of 240 trimeric hexon capsomeres and 12 pentameric penton bases. The trimeric fiber protein protrudes from the penton base at each of the 12 vertices of the capsid and is a knobbed rod-like structure. The most remarkable and obvious difference in the surface of adenovirus capsids compared to that of most other icosahedral viruses is the presence of the long, thin fiber protein (adenovirus capsid protein). The primary role of the adenovirus capsid protein is the tethering of the viral capsid to the cell surface via its interaction with a cellular receptor.

Structurally, the hexon of adenovirus capsid comprises three identical polypeptides of 967 amino acids each, namely polypeptide II [Roberts et al., Three-dimensional structure of the adenovirus major coat protein hexon, Science, 232, 1148-1151 (1986)]. The penton contains a penton base, which is bound to the capsid, and a fiber, which is noncovalently bound to and projects from the penton base. The fiber protein comprises three identical polypeptides of 582 amino acids each, namely polypeptide IV. The adenovirus serotype 2 (Ad2) penton base protein is an 8×9 nm ring-shaped complex composed of five identical protein subunits of 571 amino acids each, namely polypeptide III [Boudin et al., Isolation and characterization of adenovirus Type 2 vertex capsomer (Penton Base), Virology, 92,125-138 (1979)]. Proteins IX, VI, and IIIa are also present in the adenoviral coat and are thought to stabilize the viral capsid [Stewart et al., Image reconstruction reveals the complex molecular organization of adenovirus, Cell, 67, 145-154 (1991); Stewart et al., Difference imaging of adenovirus: bridging the resolution gap between X-ray crystallography and electron microscopy, EMBO J., 12(7), 2589-2599 (1993)].

The adenovirus (Ad) capsid protein used in the present application is derived from the capsid of adenoviruses. One type of adenovirus capsid protein is adenovirus fiber protein. The adenovirus capsid protein is typically a trimer consisting of a tail, a shaft, and a knob. The fiber shaft region is composed of repeating amino acid motifs, which are believed to form two alternating b-strands and b-bends [Green et al., Evidence for a repeating cross-f sheet structure in the adenovirus fibre, EMBO J., 2, 1357-1365 (1983)]. The overall length of the fiber shaft region and the number of 15 amino-acid repeats differ between adenoviral serotypes. For example, the Ad2 fiber shaft is 37 nm long and contains 22 repeats, whereas the Ad3 fiber is 11 mn long and contains 6 repeats. The receptor binding domain of the fiber protein is localized in the knob region encoded by the last 200 amino acids of the protein [Henry et al., Characterization of the Knob Domain of the Adenovirus Type 5 Fiber Protein Expressed in Escherichia coli, J. Virol. 68(8), 5239-5246 (1994)]. The regions necessary for trimerization are also located in the knob region of the protein [Henry et al. (1994), supra]. The adenovirus capsid or fiber protein is the main antigenic determinant of the virus and also determine the serotype specificity of the virus [Watson et al., An Antigenic Analysis of the Adenovirus Type 2 Fibre Polypeptide, J. Gen. Virol., 69, 525-535 (1988)].

HEV is a type II adenovirus (Ad), which is a non-enveloped DNA virus, with a diameter of about 70-90 nm and an icosahedral symmetry. Similarly, the capsid of HEV mainly comprises of three major structural proteins (hexon, penton, and fiber). The uniform sized virions are made up of 252 capsomers. The virus replicates in the nucleus and forms basophilic inclusions. The whole DNA genome sequence of the virus is now available. [Dhama K., Haemorrhagic enteritis of turkey's-current knowledge, Veterinary Quarterly, 37:1, 31-42, (2017)]. The current knowledge suggests it contains a linear double standard DNA of 26 Kbp, which is shorter than the genomic DNA of other adenoviruses [Jucker et al., Characterization of the hemorrhagic enteritis virus genome and the sequence of the putative penton base and core protein genes, J. Gen. Virol. 77:469-479 (1996)]. The genome consists of eight open reading frames (ORFs) in two clusters: one that includes ORFs 1, 2, 3 and 4, and other as ORFs 7 and 8 and at least 13 genes including 52K, IIIa, Penton base, pVI, Hexon, EP, 100K, pVIII, Fiber, IVa2. POL, pTP, and DBP. The genome of HEV has very low ratio of the overall G+C content (34.93%) when compared to other adenoviruses. Genes of penton, hexon, fiber, core proteins and polymerase derived from HEV have been identified and the amino acid sequences have been predicted [Pitcovski et al., The complete DNA sequence and genome organization of the avian adenovirus, hemorrhagic enteritis virus, Virol. 249:307-315, (1998)]. Recent findings revealed that ORF1, E3 and fiber genes are the key factors affecting virulence [Beach N M, Persistent infection of turkey's with an avirulent strain of turkey hemorrhagic enteritis virus. Avian Dis. 53:370-375, (2009)]. Similarly, during antigenic characterization of HEV, a total of 11 polypeptides ranging in molecular weight from 14 kDa to 97 kDa were detected. Monoclonal antibodies (MAbs) against HEV were chosen for identification of neutralizing epitopes and they were found to react with the 97 kDa hexon protein [Nazerian K. L., et al. Structural polypeptides of type II avian adenoviruses analyzed by monoclonal and polyclonal antibodies. Avian Dis. 35:572-578 (1991)]. The 97 kD polypeptide is the structural hexon protein, a monomer of the major outer capsid. Other structural proteins are the penton base protein, having a predicted size of about 50 kD and the fiber protein which anchors the penton base protein. This fiber protein consists of a tail and a globular head, which plays an important role in the first attachment of the virus to the cell receptor. The HEV fiber protein (HEVFP) used in the present application is derived from capsid of Hemorrhagic Enteritis Virus (HEV), encompassing the structural proteins including penton, hexon, and fiber proteins of the HEV capsid.

Regarding vaccination against HEV, avirulent strains of HEV has been developed as live vaccines [Sharma J M, et al., Response of specific-pathogen-free turkeys to vaccines derived from marble spleen disease virus and hemorrhagic enteritis virus. Avian Dis. 38:523-530, (1994); Pierson F W, Hemorrhagic enteritis and related infections. In: Diseases of poultry, 13th ed. Ames: Iowa State University Press, p. 237-247. (2013): Fadly A. M., Field vaccination against hemorrhagic enteritis of turkeys by a cell-culture live-virus vaccine. Avian Dis. 29:768-777, (1985)]. The cell-culture live-virus vaccine currently available to the commercial Turkey producer are effective in preventing disease outbreaks of virulent HEV: however, the vaccine by itself can induce immunosuppression in young birds, creating opportunities for opportunistic pathogens such as E. coli. In addition, the cell-culture live vaccine currently used cannot be administered in birds younger than 4 weeks of age due to the level of HE maternal antibody that can inactivate the live vaccine virus.

Recombinant adenoviral vectors and vaccines have also been developed. To date, recombinant Ads have been employed in a variety of gene therapy applications as carriers of foreign genes. [Kozarsky K. F. & Wilson J. M., Current Options in Genetics and Development 3:499-503 (1993): Li Q., et al., Assessment of Recombinant Adenoviral Vectors for Hepatic Gene Therapy, Human Gene Therapy 4(4), (2008)]. For example, vaccines including recombinant hexon protein-based subunit vaccines or recombinant virus-vectored vaccines using fowl poxvirus (FPV) expressing the native hexon of HEV have been reported. [Cardona C. J., et. al., Protection of turkey's from hemorrhagic enteritis with a recombinant fowl poxvirus (rFPV) expressing the native hexon of hemorrhagic enteritis virus. Avian Dis. 43(2):234-244, (1999)]. Pitcovski et al. [A subunit vaccine against hemorrhagic enteritis adenovirus. Vaccine. 23:4697-4702, (2005)] have developed a subunit vaccine utilizing the capsid protein (knob protein) of HEV expressed in E. coli. The usefulness of gene sequences derived from HEV as an immunogen has also been described previously through cloning in the adenoviral vector [U.S. Patent No. U.S. Pat. No. 6,663,872], or in plant based delivery systems (Tian Y., The use of transgenic tobacco as a production and delivery system for a vaccine against hemorrhagic enteritis virus of turkeys, Master Thesis, Virginia Tech, (1999), https://vtechworks.lib.vt.edu/handle/10919/9780]. These studies have shown that turkey's vaccinated with a recombinant fowl poxvirus vector expressing the native hexon protein of HEV can provide a high level of protection in turkeys from HE. However, HEV replication in the spleen was not prevented with the recombinant fowl poxvirus, suggesting that the anti-HEV antibody titer generated by vaccination wasn't sufficient to prevent virus replication likely related to the fowl poxvirus expression system used.

In the present application, the adenovirus gene sequences encoding adenovirus capsid proteins were strategically introduced into one or more genomic segments of a modified Pichinde virus to produce novel live recombinant vaccine vectors that can express foreign adenovirus antigen to induce strong humoral and cell-mediated immunity and little anti-vector immunity or immunosuppression in vivo. It was found that the present modified Pichinde viral vaccine was safe in chickens, turkeys, and pigs, with no adverse events. Further, administration of the genetically engineered PICV viral vector has the following characteristics: (1) the vector causes a limited transient infection without inducing viremia: (2) the virus does not spread/shed to pen mates when tested in multiple animal species such as mice, chickens, pigs and turkeys: (3) the recombinant viral vector upon administration is restricted to the lymphatic system causing no viremia and is cleared from the animal within 5 days of vaccination. The present disclosure demonstrates successful vaccination of the modified Pichinde virus (rP18tri-HEV-fiber-cFL) at day of age in the presence of HEV maternal antibody that protected birds against challenge given at 6 weeks of age.

In one example, a reverse genetics system includes three segments. The first genomic segment includes two coding regions, one that encodes a Z protein and a second that encodes an RNA-dependent RNA polymerase (L RdRp). The second genomic segment includes a coding region that encodes a nucleoprotein (NP), and may include at least one restriction enzyme site, such as a multiple cloning site (MCS). The third genomic segment includes a coding region that encodes a glycoprotein (GPC), and may include at least one restriction enzyme site, such as a multiple cloning site. At least one of the second and the third genomic segment further comprises a donor gene sequence, wherein the donor gene sequence encodes an adenovirus capsid protein or a fragment thereof. In embodiments, both the second and the third genomic segments each separately comprise a donor gene sequence encodes an adenovirus capsid protein or a fragment thereof. In embodiments, the adenovirus capsid protein is an adenovirus fiber protein described herein. In certain embodiments, the adenovirus is Hemorrhagic Enteritis Virus (HEV), and the adenovirus capsid protein is a HEV fiber protein (HEVFP).

A “coding region” or “donor gene sequence” is a nucleotide sequence that encodes a protein and, when placed under the control of appropriate regulatory sequences expresses the encoded protein. A “donor gene sequence” can be a foreign gene sequence derived from a different source in natural or synthetic form. As used herein, the term “protein” refers broadly to a polymer of two or more amino acids joined together by peptide bonds. The term “protein” also includes molecules which contain more than one protein joined by disulfide bonds, ionic bonds, or hydrophobic interactions, or complexes of proteins that are joined together, covalently or noncovalently, as multimers (e.g., dimers, tetramers). Thus, the terms peptide, oligopeptide, and polypeptide are all included within the definition of protein and these terms are used interchangeably. It should be understood that these terms do not connote a specific length of a polymer of amino acids, nor are they intended to imply or distinguish whether the protein is produced using recombinant techniques, chemical or enzymatic synthesis, or is naturally occurring.

The Z protein, L RdRp. NP protein, glycoprotein, and adenovirus capsid protein are those encoded by a Pichinde virus. The Z protein is a small RING-domain containing matrix protein that mediates virus budding and also regulates viral RNA synthesis. One example of a Z protein from a Pichinde virus is the sequence available at Genbank accession number ABU39910.1 (SEQ ID NO:1). The L RdRp protein is an RNA-dependent RNA polymerase that is required for viral DNA synthesis. One example of a L RdRp protein from a Pichinde virus is the sequence available at Genbank accession number ABU39911.1 (SEQ ID NO:2). The NP protein encapsidates viral genomic RNAs, is required for viral RNA synthesis, and also suppresses host innate immune responses. One example of a NP protein from a Pichinde virus is the sequence available at Genbank accession number ABU39909.1 (SEQ ID NO:3). The glycoprotein is post-translationally processed into a stable signal peptide (SSP), the receptor-binding G1 protein, and the transmembrane G2 protein. One example of a glycoprotein from a Pichinde virus is the sequence available at Genbank accession number ABU39908.1 (SEQ ID NO:4). The adenovirus capsid protein is any main exposed structural protein, encompassing the hexon, fiber, and penton of an adenovirus capsid. One example of an adenovirus capsid protein is the full-length fiber protein of turkey adenovirus 3, with the sequence available at Genbank accession number QNN94900.1 (SEQ ID NO:7). Another example of an adenovirus capsid protein is a protein comprising the knob protein, the shaft protein, and the adjacent part of the shaft protein, with the sequence set forth in SEQ ID NO:21. Yet another example of an adenovirus capsid protein is a protein comprising the Hexon protein, with the sequence set forth in SEQ ID NO:22. More examples of adenovirus capsid proteins of any functional fragment thereof could be found in U.S. Pat. No. 6,663,872, which is incorporated by reference herein in its entirety.

Other examples of Z proteins, L RdRp proteins, NP proteins, and glycoprotein include proteins having structural similarity with a protein that is encoded by a Pichinde virus, for instance, SEQ ID NO: 1, 2, 3, 4, 7, and 21-22. Structural similarity of two polypeptides can be determined by aligning the residues of the two polypeptides (for example, a candidate polypeptide and a reference polypeptide described herein) to optimize the number of identical amino acids along the lengths of their sequences: gaps in either or both sequences are permitted in making the alignment in order to optimize the number of identical amino acids, although the amino acids in each sequence must nonetheless remain in their proper order. A reference polypeptide may be a polypeptide described herein, such as SEQ ID NO:1, 2, 3, 4, 7, or 21-22. A candidate polypeptide is the polypeptide being compared to the reference polypeptide. A candidate polypeptide may be isolated, for example, from a cell of an animal, such as a mouse, or can be produced using recombinant techniques, or chemically or enzymatically synthesized. A candidate polypeptide may be inferred from a nucleotide sequence present in the genome of a Pichinde virus.

Unless modified as otherwise described herein, a pair-wise comparison analysis of amino acid sequences can be carried out using the Blastp program of the blastp suite-2sequences search algorithm, as described by Tatiana et al., [BLAST 2 SEQUENCES, a new tool for comparing protein and nucleotide sequence, FEMS Microbiol Lett, 174, 247-250 (1999)], and available on the National Center for Biotechnology Information (NCBI) website. The default values for all blastp suite-2sequences search parameters may be used, including general parameters: expect threshold=10, word size=3, short queries-on: scoring parameters: matrix=BLOSUM62, gap costs-existence: 11 extension: 1, compositional adjustments=conditional compositional score matrix adjustment. Alternatively, polypeptides may be compared using the BESTFIT algorithm in the GCG package (version 10.2, Madison WI).

In the comparison of two amino acid sequences, structural similarity may be referred to by percent “identity” or may be referred to by percent “similarity.” “Identity.” refers to the presence of identical amino acids. “Similarity” refers to the presence of not only identical amino acids but also the presence of conservative substitutions. A conservative substitution for an amino acid in a polypeptide described herein may be selected from other members of the class to which the amino acid belongs. For example, it is known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example. Lys for Arg and vice versa to maintain a positive charge; Glu for Asp and vice versa to maintain a negative charge; Ser for Thr so that a free-OH is maintained; and Gln for Asn to maintain a free —NH2.

The skilled person will recognize that the Z protein depicted at SEQ ID NO: 1 can be compared to Z proteins from other arenaviruses, including Lassa virus (073557.4), LCMV Armstrong (AAX49343.1), and Junin virus (NP_899216.1) using readily available algorithms such as ClustalW to identify conserved regions of Z proteins. ClustalW is a purpose multiple sequence alignment program for nucleic acids or proteins that produces biologically meaningful multiple sequence alignments of different sequences [Larkin et al., 2007, ClustalW and ClustalX version 2, Bioinformatics, 23(21): 2947-2948]. Using this information, the skilled person will be able to readily predict with a reasonable expectation that certain conservative substitutions to an Z protein such as SEQ ID NO:1 will not decrease activity of the polypeptide.

The skilled person will recognize that the L RdRp protein depicted at SEQ ID NO:2 can be compared to L RdRp proteins from other arenaviruses, including Lassa virus (AAT49002.1), LCMV Armstrong (AAX49344.1), and Junin virus (NP_899217.1) using readily available algorithms such as ClustalW to identify conserved regions of L RdRp proteins. Using this information, the skilled person will be able to readily predict with a reasonable expectation that certain conservative substitutions to an L RdRp protein such as SEQ ID NO:2 will not decrease activity of the polypeptide.

The skilled person will recognize that the NP protein depicted at SEQ ID NO:3 can be compared to NP proteins from other arenaviruses, including Lassa virus (P13699.1), LCMV Armstrong (AAX49342.1), and Junin virus (NP_899219.1) using readily available algorithms such as ClustalW to identify conserved regions of NP proteins. Using this information, the skilled person will be able to readily predict with a reasonable expectation that certain conservative substitutions to a NP protein such as SEQ ID NO:3 will not decrease activity of the polypeptide.

The glycoprotein depicted at SEQ ID NO:4 can be compared to glycoproteins from other arenaviruses, including Lassa virus (P08669), LCMV Armstrong (AAX49341.1), and Junin virus (NP_899218.1) using readily available algorithms such as ClustalW to identify conserved regions of glycoproteins. Using this information, the skilled person will be able to readily predict with a reasonable expectation that certain conservative substitutions to a glycoprotein such as SEQ ID NO:4 will not decrease activity of the polypeptide.

The full-length adenovirus capsid protein depicted at SEQ ID NO:7 can be compared to other adenovirus capsid proteins, e.g., any functional fragment of the full-length adenovirus capsid protein including but not limited to: main exposed structural proteins including the hexon, fiber, and penton base, all derived from adenoviruses, using readily available algorithms such as ClustalW to identify conserved regions of adenovirus capsid proteins. Using this information, the skilled person will be able to readily predict with a reasonable expectation that certain conservative substitutions to an adenovirus capsid protein such as SEQ ID NO:7 will not decrease activity of the polypeptide.

Thus, as used herein, a Pichinde virus Z protein, L RdRp protein, an NP protein, a glycoprotein, or an adenovirus capsid protein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to a reference amino acid sequence. Alternatively, as used herein, a Pichinde virus Z protein, L RdRp protein, an NP protein, a glycoprotein, or an adenovirus capsid protein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to a reference amino acid sequence. Unless noted otherwise, “Pichinde virus Z protein,” “Pichinde virus L RdRp protein,” “Pichinde virus NP protein,” and “Pichinde virus glycoprotein” refer to a protein having at least 80% amino acid identity to SEQ ID NO: 1, SEQ ID NO:2, SEQ ID NO:3, and SEQ ID NO:4, respectively. “Adenovirus capsid/fiber protein” refers to a protein having at least 80% amino acid identity to any one of SEQ ID NOs: 7 and 21-22.

A Pichinde virus Z protein, L RdRp protein, an NP protein, a glycoprotein, or an adenovirus capsid protein having structural similarity the amino acid sequence of SEQ ID NO:1, 2, 3, 4, 7, or 21-22, has biological activity. As used herein, “biological activity” refers to the activity of Z protein, L RdRp protein, NP protein, glycoprotein, or adenovirus capsid protein in producing an infectious virus particle. The biological role each of these proteins play in the biogenesis of an infectious virus particle is knows, as are assays for measuring biological activity of each protein.

In one embodiment, the NP protein may include one or more mutations. A mutation in the NP protein may result in a NP protein that continues to function in the production of infectious viral particles but has a decreased ability to suppress the production of certain cytokines by a cell infected with a Pichinde virus. A Pichinde virus that has decreased ability to suppress cytokine production is expected to be useful in enhancing an immunological response to an antigen encoded by the virus. Examples of mutations include the aspartic acid at residue 380, the glutamic acid at residue 382, the aspartic acid at residue 457, the aspartic acid at residue 525, and the histidine at residue 520. A person of ordinary skill in the art recognizes that the precise location of these mutations can vary between different NP proteins depending upon the presence of small insertions or deletions in the NP protein, thus the precise location of a mutation is approximate, and can vary by 1, 2, 3, 4, or 5 amino acids.

In one embodiment, the mutation in the NP protein may be the replacement of the aspartic acid, glutamic acid, or histidine at residues 380, 382, 457, 525, and/or 520 with any other amino acid. In one embodiment, the mutation may be the conservative substitution of the aspartic acid, glutamic acid, or histidine at residues 380, 382, 457, 525, and/or 520. In one embodiment, the mutation may be the replacement of the aspartic acid, glutamic acid, or histidine at residues 380, 382, 457, 525, and/or 520 with a glycine or an alanine. In one embodiment, the NP protein may include a mutation at one, two, three, or four of the residues 380, 382, 457, 525, or 520, and in one embodiment the NP protein may include a mutation at all five residues.

In one embodiment, the glycoprotein may include one or more mutations. A mutation in the glycoprotein may result in a glycoprotein that impairs virus spreading in vivo. Examples of mutations include the asparagine at residue 20, and/or the asparagine at residue 404. A person of ordinary skill in the art recognizes that the precise location of these mutations can vary between different glycoproteins depending upon the presence of small insertions or deletions in the glycoprotein, thus the precise location of a mutation is approximate, and can vary by 1, 2, 3, 4, or 5 amino acids.

In one embodiment, the mutation in the glycoprotein may be the replacement of the asparagine residue 20 and/or 404 with any other amino acid. In one embodiment, the mutation may be the conservative substitution of the asparagine residue 20 and/or 404. In one embodiment, the mutation may be the replacement of the asparagine residue 20 and/or 404 with a glycine or an alanine.

Proteins as described herein also may be identified in terms the polynucleotide that encodes the protein. Thus, this disclosure provides polynucleotides that encode a protein as described herein or hybridize, under standard hybridization conditions, to a polynucleotide that encodes a protein as described herein, and the complements of such polynucleotide sequences. As used herein, the term “polynucleotide” refers to a polymeric form of nucleotides of any length, either ribonucleotides or deoxynucleotides, and includes both double- and single-stranded DNA and RNA. A polynucleotide may include nucleotide sequences having different functions, including for instance coding sequences, and non-coding sequences such as regulatory sequences. A polynucleotide can be obtained directly from a natural source, or can be prepared with the aid of recombinant, enzymatic, or chemical techniques. A polynucleotide can be linear or circular in topology. A polynucleotide can be, for example, a portion of a vector, such as an expression or cloning vector, or a fragment. An example of a polynucleotide is a genomic segment.

An example of a polynucleotide encoding a Z protein is the nucleotides 85-372 of the sequence available at Genbank accession number EF529747.1 (SEQ ID NO:5), an example of a polynucleotide encoding an L RdRp protein is the complement of nucleotides 443-7027 of the sequence available at Genbank accession number EF529747.1 (SEQ ID NO:5), an example of a polynucleotide encoding an NP protein is the complement of nucleotides 1653 . . . 3338 of the sequence available at Genbank accession number EF529746.1 (SEQ ID NO:6), and an example of a polynucleotide encoding a glycoprotein protein is the nucleotides 52-1578 of the sequence available at Genbank accession number EF529746.1 (SEQ ID NO:6). It should be understood that a polynucleotide encoding a Z protein, an L RdRp protein, an NP protein, or a glycoprotein represented by SEQ ID NO: 1, 2, 3, or 4, respectively, is not limited to the nucleotide sequence disclosed at SEQ ID NO:5 or 6, but also includes the class of polynucleotides encoding such proteins as a result of the degeneracy of the genetic code. For example, the naturally occurring nucleotide sequence SEQ ID NO:5 is but one member of the class of nucleotide sequences encoding a protein having the amino acid sequence SEQ ID NO: 1 and a protein having the amino acid sequence SEQ ID NO:2. Similarly, it should be understood that a polynucleotide encoding an adenovirus capsid protein represented by SEQ ID NO:7, is not limited to the nucleotide sequence disclosed at SEQ ID NO: 15, but also includes the class of polynucleotides encoding such proteins as a result of the degeneracy of the genetic code. Other examples of polynucleotides encoding an adenovirus capsid protein or a functional fragment thereof are sequences set forth at SEQ ID NOs: 18-20. The class of nucleotide sequences encoding a selected protein sequence is large but finite, and the nucleotide sequence of each member of the class can be readily determined by one skilled in the art by reference to the standard genetic code, wherein different nucleotide triplets (codons) are known to encode the same amino acid.

In some embodiments, gene sequences of the present disclosure can be tailored for optimal gene expression. “Codon optimization” is defined as modifying a nucleic acid sequence for enhanced expression in the cells of the vertebrate of interest, e.g. avian, by replacing at least one, more than one, or a significant number, of codons of the native sequence with codons that are more frequently or most frequently used in the genes of that vertebrate. Various species exhibit particular bias for certain codons of a particular amino acid. In one embodiment, the gene sequences encoding the adenovirus capsid protein are codon optimized. As one example, the codon optimized gene sequence encoding the full length HEV Fiber Protein (HEVFP) is set forth at SEQ ID NO: 15.

As used herein, reference to a polynucleotide as described herein and/or reference to the nucleic acid sequence of one or more SEQ ID NOs can include polynucleotides having a sequence identity of at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to an identified reference polynucleotide sequence.

In this context, “sequence identity” refers to the identity between two polynucleotide sequences. Sequence identity is generally determined by aligning the bases of the two polynucleotides (for example, aligning the nucleotide sequence of the candidate sequence and a nucleotide sequence that includes, for example, a nucleotide sequence that encodes a protein of SEQ ID NO:1, 2, 3, or 4) to optimize the number of identical nucleotides along the lengths of their sequences: gaps in either or both sequences are permitted in making the alignment in order to optimize the number of shared nucleotides, although the nucleotides in each sequence must nonetheless remain in their proper order. A candidate sequence is the sequence being compared to a known sequence—e.g., a nucleotide sequence that includes the appropriate nucleotide sequence selected from, for example, SEQ ID NO:5 or 6. For example, two polynucleotide sequences can be compared using the Blastn program of the BLAST 2 search algorithm, as described by Tatiana et al., FEMS Microbiol Lett., 1999; 174: 247-250 (supra), and available on the world wide web at ncbi.nlm.nih.gov/BLAST/. The default values for all BLAST 2 search parameters may be used, including reward for match=1, penalty for mismatch=−2, open gap penalty=5, extension gap penalty=2, gap x_dropoff=50, expect=10, wordsize=11, and filter on.

In one embodiment, the second and/or third genomic segments may each independently include a “multiple cloning site” with one restriction site or more than one restriction site.

In one embodiment, the second and/or third genomic segments may each independently include a donor gene sequence that encodes an adenovirus capsid protein or a functional fragment thereof as an antigen. Thus, the second genomic segment includes the coding region encoding the nucleoprotein and may include a donor gene sequence that encodes an adenovirus capsid protein or a functional fragment thereof. Likewise, the third genomic segment includes the coding region encoding the glycoprotein and may include a donor gene sequence that encodes an adenovirus capsid protein or a functional fragment thereof. The second and third genomic segments may encode the same adenovirus capsid protein or a functional fragment thereof. In both the second genomic segment and the third genomic segment this donor gene sequence may be inserted into a restriction site present, such as a restriction site present in a multiple cloning site.

In one embodiment, the donor gene sequence may encode an adenovirus capsid protein or a functional fragment thereof that is useful as an antigen that can elicit an immune response in a subject.

In one embodiment, the adenovirus is HEV, and the adenovirus capsid protein is a HEV fiber protein (HEVFP). As one example, the donor gene sequence encoding HEVFP is the sequence available at Genbank accession number AY849321, with codon optimized for expression in avian cells (SEQ ID NO: 15). Such gene sequence codes for the trimeric fiber protein that protrudes from the penton base at each of the 12 vertices of the HEV capsid and is a knobbed rod-like structure. Another example of an adenovirus capsid protein is a protein comprising the knob protein, the shaft protein, and the adjacent part of the shaft protein, with the sequence set forth in SEQ ID NO:21. Yet another example of an adenovirus capsid protein is a protein comprising the Hexon protein, with the sequence set forth in SEQ ID NO:22. More examples of gene sequences encoding HEV capsid proteins could be found in U.S. Pat. No. 6,663,872, which is incorporated by reference herein in its entirety.

The fiber protein encoded by the donor gene sequence may be one that results in a humoral immune response, a cell-mediated immune response, or a combination thereof. In one embodiment, the fiber protein encoded by the donor gene sequence of the second and/or the third genomic segment is at least 6 amino acids in length. The fiber protein may be an antigen that is heterologous to the cell in which the donor gene sequence is expressed. The nucleotide sequence of the donor gene present on the second and/or the third genomic segment and encoding the adenovirus capsid protein can be readily determined by one skilled in the art by reference to the standard genetic code. The nucleotide sequence of a donor gene present on the second and/or the third genomic segment and encoding the adenovirus capsid protein may be modified to reflect the codon usage bias of a cell in which the adenovirus capsid protein will be expressed. The usage bias of nearly all cells in which a Pichinde virus would be expressed is known to the skilled person.

In one embodiment, the second and the third genomic segment may each independently comprise a reporter gene sequence encoding a protein that is useful as a detectable marker, e.g., a molecule that is easily detected by various methods. Examples include fluorescent polypeptides (e.g., green, yellow, blue, or red fluorescent proteins), luciferase, chloramphenicol acetyl transferase, and other molecules (such as c-myc, flag, 6×his, HisGln (HQ) metal-binding peptide, and V5 epitope) detectable by their fluorescence, enzymatic activity or immunological properties.

Pichinde virus is an arenavirus, and one characteristic of an arenavirus is an ambisense genome. As used herein, “ambisense” refers to a genomic segment having both positive sense and negative sense portions. For example, the first genomic segment of a Pichinde virus described herein is ambisense, encoding a Z protein in the positive sense and encoding a L RdRp protein in the negative sense. Thus, one of the two coding regions of the first genomic segment is in a positive-sense orientation and the other is in a negative-sense orientation. When the second and/or the third genomic segment includes a second coding region encoding an antigen, the coding region encoding the antigen is in a negative-sense orientation compared to the NP protein of the second genomic segment and to the glycoprotein of the third genomic segment.

Each genomic segment of the genetically modified Pichinde virus may also include nucleotides encoding a 5′ untranslated region (UTR) and a 3′ UTR. These UTRs are located at the ends of each genomic segment. Nucleotides useful as 5′ UTRs and 3′ UTRs are those present in Pichinde virus and are readily available to the skilled person [see, for instance, Buchmeier et al., Fields Virology. 5th ed. supra, pp. 1791-1827]. In one embodiment, a genomic segment that encodes a Z protein and an L RdRp protein includes a 5′ UTR sequence that is 5′ CGCACCGGGGAUCCUAGGCAUCUUUGGGUCACGCUUCAAAUUUGUCCAAUU UGAACCCAGCUCAAGUCCUGGUCAAAACUUGGG (SEQ ID NO:8) and a 3′ UTR sequence that is CGCACCGAGGAUCCUAGGCAUUUCUUGAUC (SEQ ID NO:9). In one embodiment, a genomic segment that encodes a NP protein or a glycoprotein includes a 5′ UTR sequence that is 5′ CGCACCGGGGAUCCUAGGCAUACCUUGGACGCGCAUAUUACUUGAUCAAAG (SEQ ID NO:10) and a 3′ UTR sequence that is 5′CGCACAGUGGAUCCUAGGCGAUUCUAGAUCACGCUGUACGUUCACUUCUU CACUGACUCGGAGGAAGUGCAAACAACCCCAAA (SEQ ID NO:11). Alterations in these sequences are permitted, and the terminal 27-30 nucleotides are highly conserved between the genomic segments.

Each genomic segment also includes an intergenic region (IGR) located between the coding region encoding a Z protein and the coding region encoding a L RdRp protein, between the coding region encoding a nucleoprotein and the at least one first restriction enzyme site, and between the coding region encoding a glycoprotein and at least one second restriction enzyme site. Nucleotides useful as an intergenic region are those present in Pichinde virus and are readily available to the skilled person. In one embodiment, an IGR sequence of a genomic segment that encodes a Z protein and an L RdRp protein includes 5′ACCAGGGCCCCUGGGCGCACCCCCCUCCGGGGGUGCGCCCGGGGGCCCCCG GCCCCAUGGGGCCGGUUGUU (SEQ ID NO:12). In one embodiment, an IGR sequence of a genomic segment that encodes a NP protein or a glycoprotein includes

(SEQ ID NO: 13)
5′GCCCUAGCCUCGACAUGGGCCUCGACGUCACUCCCCAAUAGGGGAGU
GACGUCGAGGCCUCUGAGGACUUGAGCU.

Each genomic segment of the genetically modified Pichinde virus may also include a regulatory sequence, including, but not limited to a promotor, a transcription promotor, a transcription initiation start site, a Kozak consensus sequence, a ribosome binding site, an RNA processing signal, a transcription terminator, a transcription termination site, a polyadenylation signal, or a combination thereof.

In one embodiment, the second and/or the third genomic segment may each comprise a Kozak consensus sequence around the initiation codon of the donor gene sequence encoding the adenovirus capsid protein. The genetic sequence of the Kozak consensus sequence is 5′ GCCACCATGG 3′, as set forth at SEQ ID NO: 16. In certain embodiments, the Kozak consensus sequence is located upstream of the donor gene sequence adenovirus, that is, toward the 5′ end of the coding strand for the donor gene sequence.

The present reverse genetic system based on modified Pichide virus may optionally comprise a fourth genomic segment encoding an RNA polymerase or variants thereof. Any number of RNA polymerases or variants may be used in the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. For example, the fourth genomic segment may comprise a coding region encoding bacteriophage T7 RNA polymerase, which is known as a DNA-dependent RNA polymerase that is highly specific for the T7 promoters that may be included in the second and/or the third genomic segments. One example of a T7 RNA polymerase from the fourth genomic segment of a Pichinde virus is the sequence available at Genbank accession number NP_041960.1 (SEQ ID NO:17).

In one embodiment, each genomic segment is present in a vector. In one embodiment, the sequence of a genomic segment in the vector is antigenomic, and in one embodiment the sequence of a genomic segment in the vector is genomic. As used herein, “anti-genomic” refers to a genomic segment that encodes a protein in the orientation opposite to the viral genome. For example, Pichinde virus is a negative-sense RNA virus. However, each genomic segment is ambisense, encoding proteins in both the positive-sense and negative-sense orientations. “Anti-genomic” refers to the positive-sense orientation, while “genomic” refers to the negative-sense orientation.

A vector is a replicating polynucleotide, such as a plasmid, phage, or cosmid, to which another polynucleotide may be attached so as to bring about the replication of the attached polynucleotide. Construction of vectors containing a genomic segment, and construction of genomic segments including insertion of a polynucleotide encoding an antigen, employs standard ligation techniques known in the art. See, e.g., Sambrook et al, Molecular Cloning: A Laboratory Manual., Cold Spring Harbor Laboratory Press (1989) or Ausubel, R. M., ed. Current Protocols in Molecular Biology (1994). A vector can provide for further cloning (amplification of the polynucleotide), i.e., a cloning vector, or for expression of an RNA encoded by the genomic segment, i.e., an expression vector. The term vector includes, but is not limited to, plasmid vectors, viral vectors, cosmid vectors, or artificial chromosome vectors. Typically, a vector is capable of replication in a prokaryotic cell and/or a eukaryotic cell. In one embodiment, the vector replicates in prokaryotic cells, and not in eukaryotic cells. In one embodiment, the vector is a plasmid.

Selection of a vector depends upon a variety of desired characteristics in the resulting construct, such as a selection marker, vector replication rate, and the like. Suitable host cells for cloning or expressing the vectors herein are prokaryote or eukaryotic cells.

An expression vector optionally includes regulatory sequences operably linked to the genomic segment. The term “operably linked” refers to a juxtaposition of components such that they are in a relationship permitting them to function in their intended manner. A regulatory sequence is “operably linked” to a genomic segment when it is joined in such a way that expression of the genomic segment is achieved under conditions compatible with the regulatory sequence. One regulatory sequence is a promoter, which acts as a regulatory signal that bind RNA polymerase to initiate transcription of the downstream (3′ direction) genomic segment. The promoter used can be a constitutive or an inducible promoter. The present application is not limited by the use of any particular promoter, and a wide variety of promoters are known by a skilled artisan in view of the disclosure herein. In one embodiment, a T7 promoter is used. Another regulatory sequence is a transcription terminator located downstream of the genomic segment. Any transcription terminator that acts to stop transcription of the RNA polymerase that initiates transcription at the promoter may be used. In one embodiment, when the promoter is a T7 promoter, a T7 transcription terminator is also used. In one embodiment, a ribozyme is present to aid in processing an RNA molecule. A ribozyme may be present after the sequences encoding the genomic segment and before a transcription terminator. An example of a ribozyme is a hepatitis delta virus ribozyme. One example of a hepatitis delta virus ribozyme is 5′ AGCTCTCCCTTAGCCATCCGAGTGGACGACGTCCTCCTTCGGATGCCCAGGTC GGACCGCGAGGAGGTGGAGATGCCATGCCGACCC (SEQ ID NO:14).

Transcription of a genomic segment present in a vector results in an RNA molecule. When each of the genomic segments described herein is present in a cell the coding regions and the donor gene sequence of the genomic segments are expressed and viral particles that contain one copy of each of the genomic segments are produced. The genomic segments of the reverse genetics system described herein are based on Pichinde virus, an arenavirus with a segmented genome of two single-stranded ambisense RNAs. While the ability of the reverse genetics system to replicate and produce infectious virus typically requires the presence of the ambisense RNAs in a cell, the genomic segments described herein also include the complement thereof (i.e., complementary RNA), and the corresponding DNA sequences of the two RNA sequences.

The polynucleotide used to transform a host cell optionally includes one or more marker sequences, which typically encode a molecule that inactivates or otherwise detects or is detected by a compound in the growth medium. For example, the inclusion of a marker sequence can render the transformed cell resistant to an antibiotic, or it can confer compound-specific metabolism on the transformed cell. Examples of a marker sequence are sequences that confer resistance to kanamycin, ampicillin, chloramphenicol, tetracycline, and neomycin.

Also provided herein are compositions including a viral particle described herein, or the genomic segments described herein. Such compositions typically include a pharmaceutically acceptable carrier. As used herein “pharmaceutically acceptable carrier” includes saline, solvents, dispersion media, coatings, antibacterial and antifungal agents, isotonic and absorption delaying agents, and the like, compatible with pharmaceutical administration. Additional active compounds can also be incorporated into the compositions.

A composition described herein may be referred to as a vaccine. The term “vaccine” as used herein refers to a composition that, upon administration to an animal, will increase the likelihood the recipient mounts an immune response to the adenovirus capsid protein encoded by one or more of the genomic segments described herein.

A composition may be prepared by methods well known in the art of pharmaceutics. In general, a composition can be formulated to be compatible with its intended route of administration. Administration may be systemic or local. Examples of routes of administration include parenteral (e.g., intravenous, intradermal, subcutaneous, intraperitoneal, intramuscular), enteral (e.g., oral), and topical (e.g., epicutaneous, inhalational, transmucosal) administration. Appropriate dosage forms for enteral administration of the compound of the present invention may include tablets, capsules or liquids. Appropriate dosage forms for parenteral administration may include intravenous administration. Appropriate dosage forms for topical administration may include nasal sprays, metered dose inhalers, dry-powder inhalers or by nebulization.

Solutions or suspensions can include the following components: a sterile diluent such as water for administration, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; electrolytes, such as sodium ion, chloride ion, potassium ion, calcium ion, and magnesium ion, and agents for the adjustment of tonicity such as sodium chloride or dextrose. pH can be adjusted with acids or bases, such as hydrochloric acid or sodium hydroxide. A composition can be enclosed in ampoules, disposable syringes or multiple dose vials made of glass or plastic.

Compositions can include sterile aqueous solutions (where water soluble) or dispersions and sterile powders for the extemporaneous preparation of sterile solutions or dispersions. For intravenous administration, suitable carriers include physiological saline, bacteriostatic water, phosphate buffered saline (PBS), and the like. A composition is typically sterile and, when suitable for injectable use, should be fluid to the extent that easy syringeability exists. It should be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile solutions can be prepared by incorporating the active compound (e.g., a viral particle described herein) in the required amount in an appropriate solvent with one or a combination of ingredients enumerated above, followed by filtered sterilization. Generally, dispersions are prepared by incorporating the active compound into a sterile vehicle, which contains a basic dispersion medium and any other appropriate ingredients. In the case of sterile powders for the preparation of sterile injectable solutions, preferred methods of preparation include vacuum drying and freeze-drying which yields a powder of the active ingredient plus any additional desired ingredient from a previously sterilized solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin: an excipient such as starch or lactose, a disintegrating agent such as alginic acid, Primogel, or corn starch: a lubricant such as magnesium stearate or sterotes: a glidant such as colloidal silicon dioxide: a sweetening agent such as sucrose or saccharin: or a flavoring agent such as peppermint, methyl salicylate, or orange flavoring.

For administration by inhalation, the active compounds are delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds may be prepared with carriers that will protect the compound against rapid elimination from the body, such as a controlled release formulation, including implants. Biodegradable, biocompatible polymers can be used, such as ethylene vinyl acetate, polyanhydrides, polyglycolic acid, collagen, polyorthoesters, and polylactic acid. Such formulations can be prepared using standard techniques. Liposomal suspensions can also be used as pharmaceutically acceptable carriers. These can be prepared according to methods known to those skilled in the art.

The data obtained from cell culture assays and animal studies can be used in formulating a range of dosage for use in an animal. The dosage of such compounds lies preferably within a range of circulating concentrations that include the ED₅₀ (the dose therapeutically effective in 50% of the population) with little or no toxicity. The dosage may vary within this range depending upon the dosage form employed and the route of administration utilized.

The compositions can be administered once to result in an immune response, or one or more additional times as a booster to potentiate the immune response and increase the likelihood immunity to the adenovirus capsid protein is long-lasting. A prime-boost administration regime may be used to administer the present compositions. The prime-boost administration regime may comprise at least one primary administration and at least one booster administration of the compositions. The skilled artisan will appreciate that certain factors may influence the dosage and timing required to effectively treat a subject, including but not limited to the severity of the disease or disorder, previous treatments, the general health and/or age of the subject, and other diseases present.

Also provided herein are methods for using the genomic segments. In one embodiment, a method includes making an infectious viral particle. Such a method includes, but is not limited to, providing a cell that includes each of the genomic segments described herein (a first genomic segment, a second genomic segment, a third genomic segment, and/or optionally a fourth genomic segment) and incubating the cell under conditions suitable for generating full-length genomic RNA molecules of each genomic segment. The full-length genomic RNA of each genomic segment is antigenomic. Production of full-length genomic RNA molecules of each genomic segment results in transcription and translation of each viral gene product and amplification of the viral genome to generate infectious progeny virus particles. As used herein, an “infectious virus particle” refers to a virus particle that can interact with a suitable eukaryotic cell, such as a mammalian cell (e.g., a murine cell or a human cell) or an avian cell, to result in the introduction of the genomic segments into the cell, and the transcription of the genomic segments in the cell. The method may also include introducing into the cell vectors that encode the genomic segments. Infectious virus particles are released into supernatants and may be isolated and amplified further by culturing on cells. The method may include isolating a viral particle from a cell or a mixture of cells and cellular debris. The method may include inactivating virus particles using standard methods, such a hydrogen peroxide treatment. Also provided is a viral particle, infectious or inactivated, that contains the genomic segments described herein.

In one embodiment, a method includes expression of an adenovirus capsid protein in a cell. Such a method includes, but is not limited to, introducing into a cell the genomic segments described herein, wherein one or more of the genomic segments each comprises a donor gene sequence encoding the adenovirus capsid protein. The cell is a suitable eukaryotic cell, such as a mammalian cell (e.g., a murine cell or a human cell) or an avian cell. In some embodiments, the vertebrate cell is a mammalian cell, or a human cell, or an avian cell. The cell may be ex vivo or in vivo. The genomic segments may be introduced by contacting a cell with an infectious virus particle that contains the genomic segments, or by introducing into the cell vectors that include the genomic segments. The method further includes incubating the cell under conditions suitable for expression of the coding regions present on the genomic segments, including the one or two second coding regions present on the second and/or third genomic segments. As used herein, “ex vivo” refers to a cell that has been removed from the body of an animal. Ex vivo cells include, for instance, primary cells (e.g., cells that have recently been removed from a subject and are capable of limited growth in tissue culture medium), and cultured cells (e.g., cells that are capable of long term culture in tissue culture medium). “In vivo” refers to cells that are within the body of a subject.

In one embodiment, a method includes immunizing an animal against adenovirus antigen. Such a method includes, but is not limited to, administering to an animal a viral particle that is infectious or inactivated, that contains the genomic segments described herein. The second and/or the third genomic segment includes a donor gene sequence that encodes an adenovirus capsid protein. Administration of the viral particle can result in vaccinating an animal against adenovirus antigen. The animal may be any animal in need of immunization, including a vertebrate, such as a mammal or an avian. The animal can be, for instance, avian (including, for instance, chicken or turkey), bovine (including, for instance, a member of the species Bos taurus), caprine (including, for instance, goat), ovine (including, for instance, sheep), porcine (including, for instance, swine), bison (including, for instance, buffalo), a companion animal (including, for instance, cat, dog, and horse), members of the family Muridae (including, for instance, rat or mouse), Guinea pig. In one embodiment, the animal may be an animal at risk of exposure to adenovirus infection, such as turkeys. The immune response may be a humoral response (e.g., the immune response includes production of antibody in response to an adenovirus antigen), a cellular response (e.g. the activation of phagocytes, antigen-specific cytotoxic T-lymphocytes, and the release of cytokines in response to an adenovirus antigen), or a combination thereof.

In some embodiments, the animal is at least 1 week old, 2 weeks old, 3 weeks old, 4 weeks old, 5 weeks old, 6 weeks old, 7 weeks old, 8 weeks old, 9 weeks old, 10 weeks old, 11 weeks old, 12 weeks old, 13 weeks old, 14 weeks old, 15 weeks old, 16 weeks old, 17 weeks old, 18 weeks old, 19 weeks old, 20 weeks old, 21 weeks old, 22 weeks old, 23 weeks old, 24 weeks old, 25 weeks old, 26 weeks old, 27 weeks old, 28 weeks old, 29 weeks old, 30 weeks old, 31 weeks old, 32 weeks old, 33 weeks old, 34 weeks old, 35 weeks old, 36 weeks old, 37 weeks old, 38 weeks old, 39 weeks old, or 40 weeks old. In some embodiments, the animal is at least 1 day old, 2 days old, 3 days old, 4 days old, 5 days old, 6 days old, 7 days old, 8 days old, 9 days old, 10 days old, 11 days old, 12 days old, 13 days old, 14 days old, 15 days old, 16 days old, 17 days old, 18 days old, 19 days old, 20 days old, 21 days old, 22 days old, 23 days old, 24 days old, 25 days old, 26 days old, 27 days old, or 28 days old.

In another embodiment, a method includes treating one or more symptoms or conditions of adenovirus infection or diseases associated with adenovirus in an animal. As used herein, the term “infection” refers to the presence of and multiplication of a virus or microbe in the body of a subject. The infection can be clinically inapparent or result in symptoms associated with disease caused by the virus or microbe. The infection can be at an early stage, or at a late stage. As used herein, the term “disease” refers to any deviation from or interruption of the normal structure or function of a part, organ, or system, or combination thereof, of a subject that is manifested by a characteristic symptom or set of symptoms. The method includes administering an effective amount of a composition described herein to an animal having or at risk of having adenovirus, or symptoms of adenovirus infection, and determining whether at least one symptom of the adenovirus is changed, preferably, reduced.

Treatment of symptoms associated with adenovirus can be prophylactic or, alternatively, can be initiated after the development of adenovirus. As used herein, the term “symptom” refers to objective evidence in a subject of a condition caused by adenovirus infection. Symptoms associated with adenovirus referred to herein and the evaluations of such symptoms are routine and known in the art. Treatment that is prophylactic, for instance, initiated before a subject manifest's symptoms of adenovirus infection, is referred to herein as treatment of a subject that is “at risk” of developing the adenovirus infection. Accordingly, administration of a composition can be performed before, during, or after the occurrence of adenovirus infection described herein. Treatment initiated after the development of a condition may result in decreasing the severity of the symptoms of one of the adenovirus infections or completely removing the symptoms. In this aspect of the invention, an “effective amount” is an amount effective to prevent the manifestation of symptoms of adenovirus infection, decrease the severity of the symptoms of adenovirus infection, and/or completely remove the symptoms.

Also provided herein is a kit for immunizing an animal. The kit includes viral particles as described herein, where the second and/or third genomic segments each independently include a donor gene sequence that encodes an adenovirus capsid protein, in a suitable packaging material in an amount sufficient for at least one immunization. In one embodiment, the kit may include more than one type of viral particle, e.g., the kit may include one viral particle that encodes a first adenovirus capsid protein and a second viral particle that encodes a second adenovirus capsid protein that is different from the first adenovirus capsid protein. Optionally, other reagents such as buffers and solutions needed to practice the invention are also included. Instructions for use of the packaged viral particles are also typically included.

As used herein, the phrase “packaging material” refers to one or more physical structures used to house the contents of the kit. The packaging material is constructed by known methods, preferably to provide a sterile, contaminant-free environment. The packaging material has a label which indicates that the viral particles can be used for immunizing an animal. In addition, the packaging material contains instructions indicating how the materials within the kit are employed to immunize an animal. As used herein, the term “package” refers to a solid matrix or material such as glass, plastic, paper, foil, and the like, capable of holding within fixed limits viral particles. Thus, for example, a package can be a glass vial used to contain an appropriate number of viral particles. “Instructions for use” typically include a tangible expression describing the number of viral particles, route of administration, and the like.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present disclosure is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

The present invention is illustrated by the following examples. It is to be understood that the particular examples, materials, amounts, and procedures are to be interpreted broadly in accordance with the scope and spirit of the invention as set forth herein.

### EXAMPLES

### Veterinary Vaccine Vector Carrying Donor Genes Encoding the Hemorrhagic Enteritis Virus (HEV) Full Length Fiber Protein.

As described herein, a reverse genetic system was successfully designed and developed to produce novel recombinant infectious veterinary vector, virus, and viral particles thereof specifically for the vaccination of healthy turkeys against Hemorrhagic Enteritis Virus (HEV). The veterinary viral vector was constructed based on PICV, which was used as a master seed to carry and deliver its own viral genomic segments and gene sequences thereof (encoding NP, GPC, L & Z) and a donor gene (foreign gene of interest) encoding HEVFP. Duplicate genes sequences of interest (codon optimized for avian expression) coding for the Fiber Protein of HEV (Type II Avian Adenovirus) were engineered into the cloning sites of the PICV vector. The recombinant construct was evaluated as a vaccine for controlling Hemorrhagic Enteritis (HEV) in turkey's.

The veterinary vaccine vectors described herein can induce strong humoral and cell-mediated immunity, such as a robust production of HEV-specific neutralizing antibodies and a strong HEV-specific cytotoxic T lymphocyte (CTL) response in vaccinated turkeys with little anti-PICV vector immunity. Therefore, this novel PICV-based veterinary vaccine vector system described herein satisfies all required criteria of an ideal viral vector, such as safety, induction of strong and durable cellular and humoral immune responses, no pre-existing immunity and lack of anti-vector immunity.

### Example 1

### Construction of Plasmids Encoding the Engineered Pichinde Virus P18S RNA Segments.

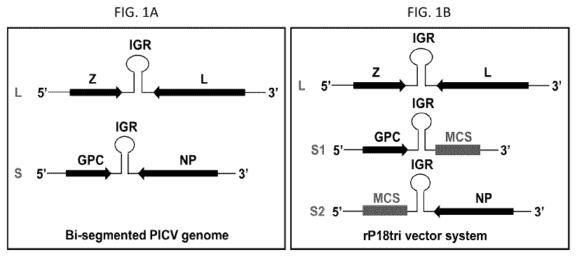

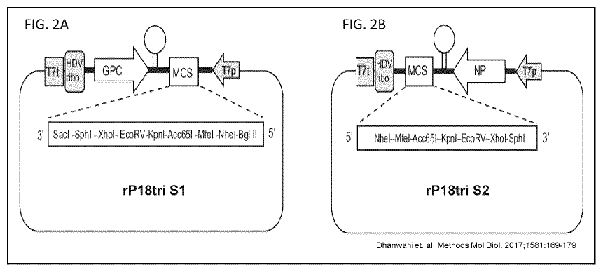

A reverse genetics system for PICV was previously developed by transfection of 2 plasmids, encoding the L and S RNA segments in anti-genomic (ag) sense, into BHK-T7 cells (FIGS. 1A and 1B) [Lan et al., J Virol 83:6357-6362, (2009), supra]. The overlapping polymerase chain reaction (PCR) method was used to replace the open-reading-frame (ORF) of either the viral glycoprotein (GPC) or nucleoprotein (NP) gene with multiple cloning sites (MCS) in the S agRNA encoding plasmid. The resulting plasmids, P18S1-GPC/MCS and P18S2-MCS/NP, as shown in FIGS. 2A and 2B, contain MCS with the restriction enzyme sequences Sac I-Sph I-XhoI-ECoRV-KpnI-Acc65I-MfeI-NheI-Bgl II and Nhe I-Mfe I-Acc65I-Kpn I-EcoR V-Xho I-Sph I, respectively, which are introduced into these plasmids for the convenience of cloning foreign genes (e.g., reporter genes and/or viral antigens).

### Example 2

Subcloning of the Donor Gene Encoding HEV Fiber Protein (HEVFP) into the P18S Vector.

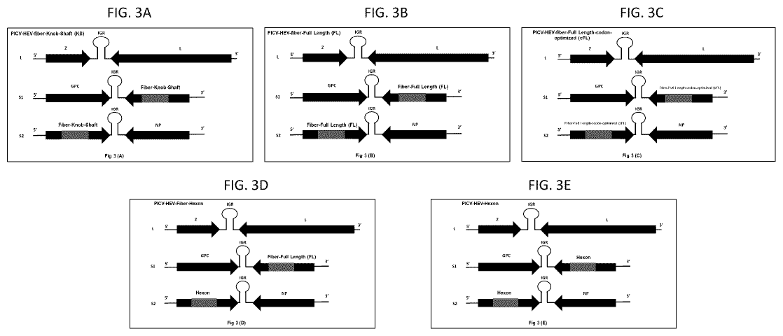

It has been previously reported to use PCR to subclone foreign genes of interest into the P18S vectors. [Dhanwani et al., J Virol 90: 2551-2560, (2015), supra]. Here, the donor gene encoding HEVFP respectively into the vectors P18S1-GPC/MCS and P18S-MCS/NP at or between Nhe I and Kpn I sites of their corresponding MCS (FIG. 3). The donor gene encoding the HEVFP (Genbank AY849321, codon optimized for expression in avian cells) was synthesized by a commercial vendor (Genescript, NJ). The HEV Fiber gene codes for the trimeric fiber protein that protrudes from the penton base at each of the 12 vertices of the capsid and is a knobbed rod-like structure. The primary role of the fiber protein is the tethering of the viral capsid to the cell surface via its interaction with a cellular receptor. The donor gene sequence encoding the full-length HEVFP is set forth in SEQ ID NO:15. The recombinant plasmid constructs were confirmed by DNA sequencing. The resulting plasmids are called rP18S1-GPC/HEVFP and rP18S2-HEVFP/NP, respectively.

It should be noted that reporter genes such as sequences encoding Green Fluorescent Protein (GFP) can also be subcloned into the P18S segments or vector to produce detectable markers.

### Example 3

### Recovery of Recombinant Veterinary Virus Expressing GPC, NP, and HEVFP.

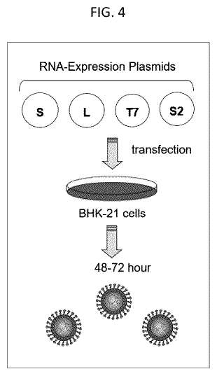

Recombinant viruses were recovered from plasmids by transfecting Baby Hamster Kidney fibroblasts (BHK-21 cells) with a plasmid expressing T7 polymerase and 3 plasmids expressing the full-length P18 L agRNA segment, the rP18S1-GPC/HEVFP segment, and the rP18S2-HEVFP/NP (FIG. 4). The procedures to generate recombinant PICV are essentially the same as previously described [Lan et al., J Virol 83:6357-6362, (2009): Dhanwani et al., J Virol 90: 2551-2560, (2015), supra). Briefly, BHK-21 cells were grown in Dulbecco's modified Eagle medium (DMEM with high glucose) supplemented with 5-10% bovine serum to 80%. The BHK-21 cells were allowed to grow to confluency and 4 hours before transfection the cells were washed and incubated with antibiotic-free media. For transfection, the BHK-21 cells were transfected with the plasmids. After 48 hours of transfection, supernatants were collected from the transfected cells and detected for infectious viruses by plaque assaying on Vero cells. Single plaques were picked from plates and amplified in BHK-21 cells to prepare viral vaccine stocks. The resulting viral vaccine was named PICV-HEVFP vaccine. Supernatants containing the rescued RBA were collected from the transfected cells at 48-72 hr time points.

### Example 4

### Gene Insertion

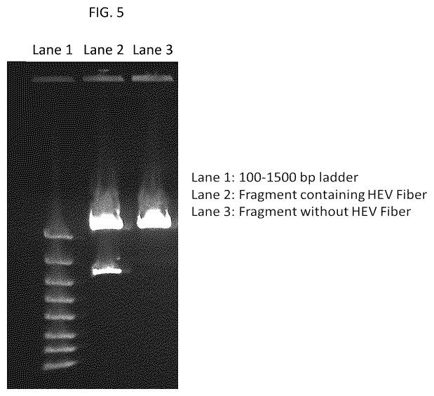

Gene insertion was confirmed by restriction digestion with Nhe I and Kpn I, giving a band of 600 bp corresponding to the HEVFP segment. The plasmid was sequenced to confirm the inserted nucleotide sequence. As shown in FIG. 5, restriction digestion with NheI and ScaI resulted in two bands of 4.3 kb and 1 kb, respectively. PCR product of 1.2 kb, using a single forward (ACACATTCTGGTGCCCTTAC) and a reverse primer (ACCCACTATTTGGAGCAAGC), defines the insert and PICV specific sequences. Restriction digestion, PCR pattern, and complete viral genome sequencing were used to test Master Seed (n) and (n+5) and confirmed the stability and purity of the veterinary viral vectors.

The rP18tri-segment based viruses form smaller plaques in cell culture than the parental P18 virus, illustrating a growth attenuation of the recombinant virus carrying the tri-segmented genome [Dhanwani, et al., J Virol, 90(5):2551-2560, (2015), supra]. It was further showed that the tri-segmented PICV is highly attenuated in vivo. No adverse events were detected in mice, chickens, pigs, and turkeys. Only transient infection was observed with no clinical signs detected. The full-length HEVFP gene does not provide any new virulence factors to the PICV backbone, the recommended biosafety level will be the same as the parent PICV, with no expected safety concerns for any animal species. Further, there were no genetic motifs that have been detected that would promote homologous recombination, DNA insertion, or gene expression of existing or new open reading frames of the PICV segments. It is therefore expected that the present PICV-HEVFP viral vaccine will not cause clinical signs in animals administered live vaccine.

### Example 5

### Experimental Overview

Our overall goal in the following Examples 5-13 is to develop a vaccine for controlling Hemorrhagic Enteritis virus (HEV) in turkey's utilizing the tri-segmented Pichinde viral vector. We evaluated multiple capsid proteins to include the fiber knob protein, together with an adjacent part of the shaft domain (SEQ ID NO:18); the hexon protein (SEQ ID NO: 19), the full-length fiber protein (SEQ ID NO:20) and the codon optimized full-length protein (SEQ ID NO:15) as vaccine targets. Table 1 shows the various Pichinde virus that encode different HEV Fiber Proteins. These Pichinde virus were prepared following the methods provided in Examples 1-4.

TABLE 1
Various Pichinde viruses used in the present studies.

| | | Gene sequence | |
|---|---|---|---|
| | | Encoding the | Amino acid sequence |
| | | HEV Fiber | of the HEV Fiber |
| Pichinde Virus | HEV Fiber Protein | Protein | Protein |
| PICV-HEV-fiber-Full Length (FL) | Full-length | SEQ ID NO: 20 | SEQ ID NO: 7 |
| PICV-HEV-fiber-Full Length-codon- | Full-length | SEQ ID NO: 15 | SEQ ID NO: 7 |
| optimized (cFL) | | | |
| PICV-HEV-fiber-Knob-Shaft (KS) | Knob + Shaft | SEQ ID NO: 18 | SEQ ID NO: 21 |
| PICV-HEV--Fiber-Hexon | Hexon | SEQ ID NO: 19 | SEQ ID NO: 22 |

### Justification

In 2020, approximately 229 million turkeys were raised on about 2,500 farms across the United States generating over 4.3 billion dollars annually. The majority of commercial turkeys across the United States are vaccinated with a live HEV vaccine at three to four weeks of age. Vaccination has been shown to reduce the spread and incidence of virulent Hemorrhagic Enteritis virus. Nevertheless, the commercially available live HEV vaccines on the market today have been shown to cause immune suppression creating opportunities for opportunistic secondary infections particularly with E. coli leading to high flock morbidity, mortality and carcass condemnation at processing.

The utilization of the tri-segmented Pichinde vector expressing foreign protein antigens of HEV may have a number advantages over the commercially available live HE vaccines on the market today 1) day of age hatchery vaccination in the presence of HE maternal antibody 2) more uniform and consistent vaccination at day of age in contrast to field application 3) ability to give a booster vaccination without neutralizing the vector and 4) vaccinating without causing immunosuppression. These advantages would be highly receptive to the Turkey industry to help manage HEV as an etiologic agent.

### Preparation of Virulent Challenge Strain of Hemorrhagic Enteritis Virus (HEV)

The virulent strain of HE virus (HEV) used in the following experiments was an original field isolate derived from infected splenic tissue of turkey's that had died of Hemorrhagic Enteritis. Briefly, 20 spleens of dead turkeys showing severe clinical signs of splenomegaly were removed, pooled together and frozen at −80° C. until processed.

Spleens were thawed in a laminar flow hood at room temperature and homogenized in a tissue homogenizer by placing 5 spleens in a 20% wt/vol suspension in phosphate-buffered saline (PBS). The homogenized tissue suspension was frozen and thawed three times and centrifuged at 800×g then at 2,000×g to remove cellular debris. The clarified splenic suspension as described above was then extracted by adding trichlorotrifluorethane at a ratio of 1:3. The mixture was blended, centrifuged at 2,000×g at 4° C. for 5 min and the supernatant was kept on ice, and the pellet was reextracted. The suspension was then filtered through a 0.45 μm syringe disc filter. The pooled supernatants were concentrated to 25% of the original volume by dialysis against dry polyethylene glycol (PEG).

The concentrated material was then layered onto sucrose-cesium chloride (CsCl) gradients. The gradients were prepared by overlaying 4 ml samples of 35 and 40% CsCl and 2

ml of IM sucrose in cellulose nitrate tubes. Both the CsCl and sucrose solutions were made in 50 mM Tris, pH 8.0. The gradients were centrifuged using a Beckman Ultra-centrifuge at 100,000×g for 4 hours at 4 C. Following centrifugation, the virus was visualized by indirect light and collected by inserting a 22-gauge needle attached to a 3 ml lure-lock syringe through the side of the cellulose tube just below the viral band. The density of the collected fraction was determined with a refractometer. The buoyant density of the viral fraction was 1.34 g/cm³, typical for adenoviral particles. The final suspension was aliquoted into 1.0 ml samples and frozen in 2 ml cryogenic vials and stored frozen at −80° C. until use.

The purity of the virus was examined by Transmission Electron Microscopy (TEM). Briefly, a 100 ul sample of the purified viral fraction was nebulized onto carbon-coated Formvar film grids, washed briefly in distilled water by placing the grid onto the droplet of water, stained with 2%, phosphotungstic acid (PTA) and examined in a JEOL 100CX electron microscope at 80 kV.

### Example 6

### Preparation of Challenge Vaccine

The purified virus of Example 5 was expanded in 1) live Specific Pathogen Free (SPF) turkeys and 2) cell culture using a lymphoblastoid cell lines designated as MDTC-RP19, which was previously established from tumors induced by Marek's disease virus in turkeys.

### Virus Propagation in Live SPF Turkeys

Briefly, 100 SPF turkey poults were raised in isolation from 1 day of age to 6 weeks. Virus propagation was done by diluting 1 ml of the stock virus (of example 5) into 99 ml of cold PBS pH 7.2 to give a 1: 100 dilution. Turkeys were intravenously inoculated and at five days post killed by cervical dislocation. The spleens were removed weighed, and scored as positive or

negative for virus infection on the basis of splenic enlargement. Large spleens were pooled into groups of ten and frozen at −80° C. until use.

### The Concentration of Virus in Infected Spleens

Briefly, the spleens as described above were thawed, homogenized and adjusted to a 20% suspension prepared in cold PBS. The suspension was centrifuges at 3000×g to remove cellular debris and the resulting supernatant was collected and filtered through a 0.45-disc filter. The RP19 cells cultured in 75 cm² flask were infected with 5 ml of the HEV splenic supernatant, the cells were maintained in 1:1 serum-reduced Lei-bovitz's-L15-McCoy's 5A media, supplemented with 2.5% fetal bovine serum (FBS), 5% CS, 1.2% tryptose phosphate broth (TPB) (ThermoFisher Scientific). Cell enlargement was observed in the infected cultures at 48 hours. Virus titration was done by using ten-fold serial dilutions of the virus in the RP19 cells. The end point concentration of the viral stock was then determined by the highest dilution showing cell enlargement. The culture supernatant of the enumerated viral stock was collected, and aliquoted into 1.0 ml samples and frozen in 2 ml cryogenic vials and stored frozen at −80° C. until use.

### Expansion and Enumeration of Virulent HEV in RP19 Cell Culture

Tissue-culture propagated virulent HEV was done in RP19 cells of Example 5 by growth in suspension culture in 1:1 complete Lei-bovitz's L15 and McCoy's 5A (CLM) media (ThermoFisher Scientific) at a 1:1 ratio, supplemented with 10% fetal bovine serum, 20% chicken serum, 5% tryptose phosphate broth incubated at 41° C. in a humidified atmosphere with 5% CO2. Virus titration was done by using ten-fold serial dilutions of the purified viral stock of Example 5 in the RP19 cells. The enumerated viral stock was collected, and aliquoted into 1.0 ml samples and frozen in 2 ml cryogenic vials and stored frozen at −80° C. until use.

### Example 7

### HEV Challenge Optimization in 7-Week Old Turkeys

The HEV virus stocks prepared in RP19 cells was tested for pathogenicity in 7-week old turkeys to optimize the challenge dose of the HEV stocks. Briefly, one hundred thirty-six (N=136) turkeys were divided equally into 6 groups (21 turkeys/group) with the exception of Group 7 having only 10 birds designated as 1-7. Group 7 acted as a non-challenged control. At seven weeks of age all turkeys in groups 1-6 were challenged with the designated dose & route as described in Table 2. At seven and ten-days post challenge 7 birds from each group (1-6) were exsanguinated by cervical dislocation, necropsied and spleens were removed and weighed to determine the TCID₅₀ as seen by splenomegaly. Splenomegaly is calculated by determining the weight of each spleen divided by the weight of the bird to determine the spleen to body weight ratio. In addition, the jejunum of each bird was examined for Petechial hemorrhage Table 3.

TABLE 2
Experimental Design

| | | | Inoculation | Inoculation |
|---|---|---|---|---|
| Groups | Turkeys | HEV stock | Volume | Route |
| 1 | 21 | Spleen homogenate low dilution | 0.5 ml | Oral |
| 2 | 21 | Spleen homogenate High | 0.5 ml | Oral |
| | | dilution | | |
| 3 | 21 | Spleen homogenate low dilution | 0.5 ml | IV |
| 4 | 21 | HEV new stock 10\hat{ }6 TCID₅₀ | 0.5 ml | Oral |
| 5 | 21 | HEV new stock 10\hat{ }4 TCID₅₀ | 0.5 ml | Oral |
| 6 | 21 | HEV new stock 10\hat{ }4 TCID₅₀ | 0.5 ml | IV |
| 7 | 10 | Mock (Tissue culture medium) | 0.5 ml | Oral |

TABLE 3
Experiment Schedule

| Day | Date | Procedure |
|---|---|---|
| 0 | Oct. 2, 2018 | 136 turkeys placed. |
| 49 | Nov. 20, 2018 | HEV challenge Oral & IV. |
| 56 | Nov. 27, 2018 | Seven days Post Challenge 7 birds from groups 1-6 were necropsied and |
| | | Spleen was collected, weighed & stored in Tissue culture medium. The |
| | | jejunum of each bird was examined for Petechial hemorrhage. |
| 59 | Nov. 30, 2018 | Ten days Post Challenge 7 birds from groups 1-6 were necropsied and |
| | | Spleen was collected, weighed & stored in Tissue culture medium. The |
| | | jejunum of each bird was examined for Petechial hemorrhage. |

At seven days post HEV challenge the results showed that groups 1, 2 and 5 given orally induced the highest degree of splenomegaly in contrast to all other test groups except for birds in Group 1 which acted as the non-challenged control group. It was also shown that intestinal hemorrhage was also most prominent in these same groups (data not shown) upon post mortem examination, in contrast to the challenge given intravenously. These results reconfirm that the natural progression of disease is by fecal oral transmission in which infected birds peck fecal material that has been shed by pen mates excreting virus in their feces. In addition, the peak of infection was seen at day-7 in contrast to day-10 seen by the lack of splenomegaly and intestinal hemorrhages (data not shown) in all test groups. Thus, the HEV TCID₅₀ at 10⁴ was used for the following experimental challenge studies.

### Example 8

Generating the rP18tri Vectors Expressing Hemorrhagic Enteritis Virus Antigens (Fiber-Full Length, Hexon and Hexon/Fiber Protein)

### Cells and Viruses:

BHK-21 baby hamster kidney cells were maintained in Dulbecco's modified Eagle medium (DMEM) supplemented with 10% fetal bovine serum (FBS), Vero African green monkey kidney cells, cells were maintained in minimum Essential medium (MEM) supplemented with 10% fetal bovine serum (FBS). Recombinant PICVs were plaque purified and were amplified in BHK-21 cells, and the infectious virus titer was determined by a plaque assay in Vero cells as described previously [Dhanwani R., et. al, A novel live Pichinde virus-based vaccine vector induces enhanced humoral and cellular immunity upon a booster dose. J Virol 90(5):2551-2560, (2015)].

The plasmids expressing the full-length antigenomic strands of the PICV L and S segments from the T7 promoter, pUC18-P18Lag and pUC18-P18Sag, have been described previously (23). The overlapping PCR method was used to replace the GPC and NP open reading frames (ORFs) on the pUC18-P18Sag vector with multiple cloning sites (MCS), generating P18S-GPC/MCS (the S1 plasmid) and P18S-MCS/NP (the S2 plasmid), respectively. The HEV Fiber gene was amplified by PCR and was subcloned into the S1 & S2 plasmids between the NheI and KpnI sites to generate the P18S-HEV Fiber plasmids.

Rescue of Recombinant Tri-Segmented Pichinde Viruses (rP18tri) from Plasmids by Transfection:

Recombinant viruses were recovered from plasmids by transfecting BHK-21 cells with 4 plasmids: pUC18-P18Lag, P18S1-HEV Fiber, P18S2 HEV Fiber, and pCAGGS T7 plasmid. The procedures used to generate recombinant PICV are essentially the same as those described previously (23). Briefly, BHK-21 cells were grown overnight to 80% confluence, and 4 hr before transfection, the cells were washed and were incubated with antibiotic-free DMEM (Sigma-Aldrich). For transfection, 2 μg of each plasmid was diluted in 250 μl of Opti-MEM (Invitrogen-Life Technologies) and was incubated at room temperature for 15 min. An equal volume of Opti-MEM with 10 μl of Lipofectamine (Invitrogen-Life Technologies) was added, and the mixture was incubated for 20 min at room temperature. Following incubation, the cells were transfected with the plasmids, and a fresh aliquot of DMEM was added after 4 hr to remove the Lipofectamine. After 48 hr of transfection, cell supernatants were collected for a plaque assay. Virus grown from individual plaques was used to prepare stocks that were grown on BHK-21 cells and were stored at −80° C.

### Example 9

### The Efficacy of the Hexon, Hexon/Fiber and Fiber Proteins Against Hemorrhagic Enteritis Virus in Turkeys

The purpose of this experiment was to evaluate the protective efficacy of three Pichinde vectors having the gene sequences coding for the Fiber protein (full length), the hexon and the hexon/fiber protein as described in Example 5. The vaccine vectors were designated as 1) rP18tri-HEV-hexon 2) rP18tri-HEV-fiber-(FL) and 3) rP18tri-HEV-hexon/fiber. The outcome parameters used to evaluate vaccine efficacy were 1) evaluate the efficacy of each vector individually against an HE challenge compared to mock vaccinated rP18tri-GFP vector control, 2) titration of the vector dose against challenge and 3) compare the efficacy of each vector to the efficacy of the HE commercial vaccine (Oralvax) given as a single dose at 3-weeks of age.

Briefly, 150-day old turkeys poults obtained from Select Genetics (Willmar, MN) were leg banded for identity and equally divided into 10 groups (15 birds per group) designated as Groups 1-10 (Table 4). Table 4 shows the experimental design. The study consisted of ten groups (15 birds per group) designated as 1 thru 10. All birds were vaccinated at 21 days of age using a 1.0 ml volume with their appropriate vaccine. Birds in Group-1 were vaccinated with 1.0 ml of the rP18tri-GFP vector that acted as the vector control. Birds in Groups 2-9 were vaccinated with their appropriate vector at 10⁶, 10⁷, and/or 10⁸ PFU respectively. Birds in Group-10 were separated into a separate isolation room and orally gavaged with the live Oralvax HE vaccine at the manufactures recommended dose. Twenty-one days after vaccination (day-42) birds in Group-10 were transported and co-mingled back into groups 1-9. Blood was taken twenty-four hours pre-challenge from 7 birds per group and serum was collected from coagulated blood and stored at −80° C. All birds were then challenged by oral gavage with the virulent HEV at 1.0×10⁴ TCID₅₀ in a 1.0 ml volume.

TABLE 4
Experimental design.

| | | | Vaccine | Vaccination/ | |
|---|---|---|---|---|---|
| Group | Turkey | Vaccine | Volume | Route | Challenge |
| 1 | 15 | Vector control - rP18tri-GFP | 1.0 ml | IM (Day 21) | Day-42 |
| | | 10⁷ PFU vaccinated/HEV | | | |
| | | Challenged | | | |
| 2 | 15 | rP18tri-HEV-fiber-(FL) 10⁶ | 1.0 ml | IM (Day 21) | Day-42 |
| | | PFU | | | |
| 3 | 15 | rP18tri-HEV-fiber-(FL) 10⁷ | 1.0 ml | IM (Day 21) | Day-42 |
| | | PFU | | | |
| 4 | 15 | rP18tri-HEV-fiber-(FL) 10⁸ | 1.0 ml | IM (Day 21) | Day-42 |
| | | PFU | | | |
| 5 | 15 | rP18tri-HEV-hexon 10⁶ PFU | 1.0 ml | IM (Day 21) | Day-42 |
| 6 | 15 | rP18tri-HEV-hexon 10⁷ PFU | 1.0 ml | IM (Day 21) | Day-42 |
| 7 | 15 | rP18tri-HEV-hexon 10⁸ PFU | 1.0 ml | IM (Day 21) | Day-42 |
| 8 | 15 | rP18tri-HEV-hexon/fiber-(FL) | 1.0 ml | IM (Day 21) | Day-42 |
| | | 10⁶ PFU | | | |
| 9 | 15 | rP18tri-HEV-hexon/fiber-(FL) | 1.0 ml | IM (Day 21) | Day-42 |
| | | 10⁸ PFU | | | |
| 10 | 15 | Oralvax | 1.0 ml | Oral (Day 21) | Day-42 |

### Example 10

### Virulent HEV Challenge

At 21 days post vaccination (day-42) all birds were then challenged by oral gavage with the virulent HEV at 1.0×10⁴ TCID₅₀ in a 1.0 ml volume. Morbidity and Mortality was monitored once daily for 7 consecutive days. At seven days post challenge, individual birds were weighted and then terminated. All birds were necropsied to evaluate vaccine efficacy based on the lack of splenomegaly and intestinal lesions of the duodenum.

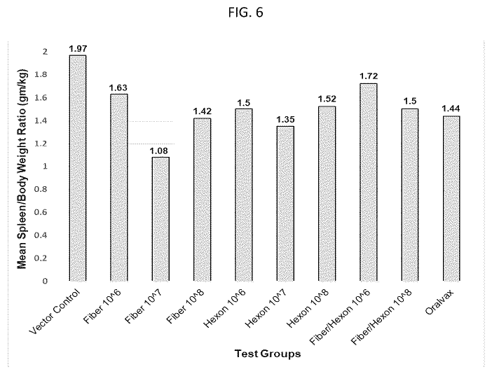

FIG. 6 shows the mean spleen/Body weight ratio of vectors rP18tri-HEV-hexon, rP18tri-HEV-fiber-(FL) and rP18tri-HEV-hexon/fiber after HEV challenge compared to the vector control (rP18tri-GFP) and Oralvax. In this study, all birds were immunized once at 21-days of age with their appropriate vaccine (Table 2). Vectors rP18tri-HEV-hexon, rP18tri-HEV-fiber-(FL) and rP18tri-HEV-hexon/fiber were tested at three different doses (1.0×10⁶ PFU, 1.0×10⁷ PFU and/or 1.0×10⁸ PFU). All birds were challenged 21-days after vaccination (day 42) by oral gavage with the virulent HEV at 1.0×10⁷ TCID₅₀. Efficacy: as measured by the lack of splenomegaly was compared to the rP18tri-GFP vector control and to Oralvax: the commercial vaccine product.

The vaccine vector, rP18tri-HEV-fiber-(FL) having the gene sequence coding for the fiber protein (full length) was the only construct that induced protective immunity as seen by the decrease in spleen/body weight ratio compared to all other test groups including Oralvax. The spleen/body weight ratio of the rP18tri-HEV-fiber-(FL) was significantly lower (1.08) compared to the vector control (1.97) and Oralvax (1.44) respectively at a single dose of 1.0×10⁷ PFU (FIG. 6). Taken together, these data show that the rP18tri-G/H viral vector induced protective immunity against an HEV challenge in turkey's as seen by the decrease in splenomegaly. It is interesting to note the lack of efficacy of rP18tri-HEV-hexon and rP18tri-HEV-hexon/fiber at multiple doses given.

### Example 11

The Efficacy of rP18tri-HEV-Fiber-(FL) Codon Optimized to Avian Species

To further test the efficacy of the full-length fiber protein the gene sequence was codon optimized to the host species (avian) to help improve gene expression and increase the translational efficiency of the gene of interest, in this case, the full-length fiber protein designated as rP18tri-HEV-fiber-(cFL). Thus, the overall goal in codon optimizing was to see if the effective dose of the vaccine could be lowered below the 1.0×10⁷ PFU, which was the dose that showed protection in the previous study of Example 9. Lowering the effective dose would make the PICV HE vaccine product more economically feasible to manufacture.

Briefly, 140-day old turkeys poults obtained from Select Genetics (Willmar, MN) were leg banded for identity and equally divided into 7 groups (20 birds per group) designated as Groups 1-7: (Group-1 were non-vaccinated/non-challenged and acted as true controls, Group-2 were Vector control-rP18tri-GFP 10⁷ PFU vaccinated/HEV Challenged, Group-3 were rP18tri-HEV-fiber-(cFL) 10⁴ PFU, Group-4 rP18tri-HEV-fiber-(cFL) 10⁵ PFU, Group-5 rP18tri-HEV-fiber-(cFL) 10⁶ PFU and Group-6 rP18tri-HEV-fiber-(cFL) 10⁷ PFU). Birds in Group-7 were separated into a separate isolation room and orally gavaged with the live Oralvax HE vaccine at the manufactures recommended dose (Table 5). The study consisted of seven groups (20 birds per group) designated as 1 thru 7. Birds in Group-1 acted as a true control (non-vaccinated/non-challenged, Birds in Group-2 were vaccinated with 1.0 ml of the rP18tri-GFP vector that acted as the vector control. Birds in Groups 3 thru 6 were vaccinated at 28 days of age using a 1.0 ml volume of their appropriate vaccine at 10⁴, 10⁵, 10⁶ and 10⁷ PFU respectively. Birds in Group-7 were separated into a separate isolation room and orally gavaged with the live Oralvax HE vaccine at the manufactures recommended dose on day-28. Twenty-one days after vaccination (day-49) birds in Group-10 were transported and co-mingled back into groups 1-6. Blood was taken twenty-four hours pre-challenge from 7 birds per group and serum was collected from coagulated blood and stored at −80° C. All birds were then challenged by oral gavage with the virulent HEV at 1.0×10⁴ TCID₅₀ in a 1.0 ml volume.

TABLE 5
Experimental design.

| | | | Vaccine | | |
|---|---|---|---|---|---|
| Group | Turkey | Vaccine | Volume | Vaccination/Route | Challenge |
| 1 | 20 | Control non-vaccinated/non- | None | None | None |
| | | challenged | | | |
| 2 | 20 | Vector control - rP18tri-GFP | 1.0 ml | IM (Day 28) | Day-49 |
| | | 10⁷ PFU vaccinated/HEV | | | |
| | | Challenged | | | |
| 3 | 20 | rP18tri-HEV-fiber-(cFL) 10⁴ | 1.0 ml | IM (Day 28) | Day-49 |
| | | PFU | | | |
| 4 | 20 | rP18tri-HEV-fiber-(cFL) 10⁵ | 1.0 ml | IM (Day 28) | Day-49 |
| | | PFU | | | |
| 5 | 20 | rP18tri-HEV-fiber-(cFL) 10⁶ | 1.0 ml | IM (Day 28) | Day-49 |
| | | PFU | | | |
| 6 | 20 | rP18tri-HEV-fiber-(cFL) 10⁷ | 1.0 ml | IM (Day 28) | Day-49 |
| | | PFU | | | |
| 7 | 20 | Oralvax | 1.0 ml | Oral (Day 28) | Day-49 |

### Example 12

### Virulent HEV Challenge

At 21 days post vaccination (day-49) birds in Groups 2-7 were challenged by oral gavage with the virulent HEV at 1.0×10⁴ TCID₅₀ in a 1.0 ml volume. Birds in Group-1 remained as non-vaccinated/non-challenged. Morbidity and Mortality was monitored once daily for 7 consecutive days. At seven days post challenge, individual birds were weighted and then terminated. All birds were necropsied to evaluate vaccine efficacy based on the lack of splenomegaly and intestinal lesions of the duodenum.

To help improve gene expression and potentially increase the translational efficiency of the gene of interest, the gene sequence of the full-length HEV fiber protein was codon optimized to the host species in avian cells and commercially custom-synthesized, and cloned into a pUC57 vector (Genescript, NJ). Thus, our goal in codon optimizing was to see if the effective dose of the vaccine could be lowered below the infective dose of 1.0×10⁷ PFU, which was the dose that showed the highest degree of protection in the previous study of Example 9. Lowering the effective dose would make the PICV-HEV vaccine product more economically feasible to manufacture. The results confirmed that codon optimizing the gene sequence indeed lowered the effective dose.

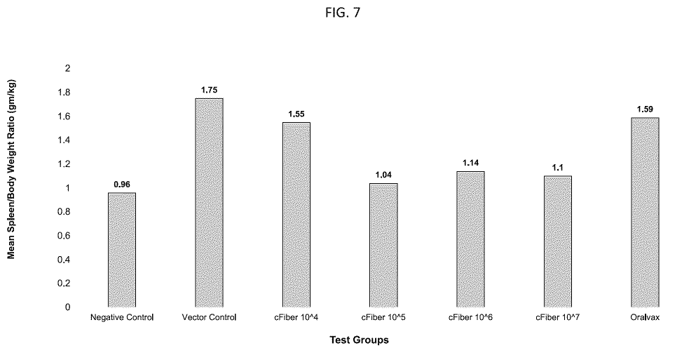

FIG. 7 shows the comparative spleen/Body weight ratio of the codon optimized vector rP18tri-HEV-fiber-cFL at different doses compared to the control and Oralvax. Please note: that the lowest infective dose of 1.0×10⁵ PFU showed the highest degree of efficacy with a mean spleen/Body weight ratio of 1.04, in contrast to the previous experiment of Example 9 which had a spleen/Body weight ratio of 1.08 using a dose of 1.0×10⁷ PFU (non-codon optimized). However, there was no statistical difference in the overall efficacy between the non-codon optimized dose compared to the codon optimized dose. Nevertheless, these results clearly illustrate that codon optimizing the gene sequence to the host species lowered the infective dose to 1.0×10⁵ PFU compared to 1.0×10⁷ PFU respectively. This is a hundred-fold difference in the infective dose required for protective efficacy of the PICV rP18tri-HEV-fiber-(cFL) vector in contrast to the non-codon optimized construct as shown in Example 9.

In terms of commercial application this is a huge cost savings in viral manufacturing as it lowers the amount of virus that would need to be produced to achieve efficacy. For example, in a large-scale production strategy using a 400 liter Bio-reactor for viral growth and the effective dose is 1.0×10⁷ PFU per 1.0 ml and the market demand is 300,000,000 doses would require 3.0×10¹⁵ PFU total. This would require an antigen harvest titer at 1.0×10⁸ PFU/ml which would require 30,000,000 ml of virus which would require 75-400 liter (30,000 liters) Bioreactor runs. Using the same scenario but now calculating for the codon optimized dose at 1.0×10⁵ PFU per dose with the same marketing demand of 300,000,000 doses would require 3.0×10¹³ PFU total. This would then require an antigen harvest titer at 1.0×10⁸ PFU/ml which would require 300,000 ml of virus which is 1-400 liter (300 liters) Bio-reactor run. Table 6 shows the summary of direct manufacturing costs of the non-codon optimized dose at 1.0×10⁷ PFU at 300 million Doses.

TABLE 6
Direct cost of non-codon optimized dose at 1.0 × 10⁷ PFU at 300 million Doses

| | Item | Quantity Used | Unit | Cost/Unit | Total cost |
|---|---|---|---|---|---|
| Cell Scale Up | DMEM Media | 4,500,000 | mL | $0.04 | $186,750.00 |
| | Calf Serum(5%) | 225,000 | mL | $0.34 | $77,175.00 |
| | Corning 1750 cm2 roller bottle | 11,250 | Ea | $27.70 | $311,625.00 |
| | Trypsin | 900,000 | mL | $0.09 | $79,020.00 |
| | Dulbeccos PBS | 900,000 | mL | $0.04 | $32,859.00 |
| | Centrifuge Tube 250 mL | 3,750 | mL | $3.32 | $12,450.00 |
| | Overhead Hrs | 9,000 | Hrs | | $0.00 |
| | 2 operators - 8-12 hours | | | | |
| | | | | Total | $699,879.00 |
| Infect/Incubation | Cytodex 3 microcarrier beads | 150,000 | g | $5.38 | $807,000.00 |
| | DMEM Media | 30,000,000 | mL | $0.04 | $1,245,000.00 |
| | Calf Serum(5%) | 1,500,000 | mL | $0.34 | $514,500.00 |
| | Overhead Hrs | 4,800 | Hrs | | $0.00 |
| | | | | Total | $2,566,500.00 |
| | 2 operators - 12-16 hours | | | | |
| Harvest | 5 L Biocontainer | 6,000 | Ea | $53.00 | $318,000.00 |
| | Sucrose | 3,000,000 | mL | $0.04 | $120,000.00 |
| | Overhead Hrs | 600 | Hrs | | $0.00 |
| | 2 operators - 4-8 hours depending on if diafiltered | | | Total | $438,000.00 |
| | and/or stabilized | | | | |

| | Item | Quantity Produced | Unit | Cost/Unit | Total cost |
|---|---|---|---|---|---|
| Final Antigen | Pichinde Antigen (1.0 × 10^{8.0} PFU/mL) | 30,000,000 | mL | 0.12 | $3,704,379.00 |

The difference in the overall cost savings using the same scenario described above taking into consideration the cost of goods for manufacturing the non-codon optimized dose compared to the codon optimized dose at a cost per unit of 0.12 is dramatic. For example, looking at the cost for basic manufacturing in terms of cell scale-up, infect/incubation, viral harvest and final antigen required to manufacture 300 million doses is 3,704,379.00 dollars of the non-codon optimized dose compared to 37,059.30 dollars to manufacture the codon optimized dose. This is a huge cost saving in manufacturing the PICV rP18tri-HEV-fiber-(cFL) vector vaccine. Table 7 shows the direct cost of codon optimized dose at 1.0×10⁷ PFU at 300 million Doses.

TABLE 7
Direct cost of codon optimized dose at 1.0 × 10⁵ PFU at 300 million Doses

| | Item | Quantity Used | Unit | Cost/Unit | Total cost |
|---|---|---|---|---|---|
| Cell Scale Up | DMEM Media | 45,000 | mL | $0.04 | $1,867.50 |
| | Calf Serum(5%) | 2,250 | mL | $0.34 | $771.75 |
| | Corning 1750 cm2 roller bottle | 113 | Ea | $27.70 | $3,130.10 |
| | Trypsin | 9,000 | mL | $0.09 | $790.20 |
| | Dulbeccos PBS | 9,000 | mL | $0.04 | $328.59 |
| | Centrifuge Tube 250 mL | 38 | mL | $3.32 | $126.16 |
| | Overhead Hrs | 120 | Hrs | | $0.00 |
| | 2 operators - 8-12 hours | | | | |
| | | | | Total | $7,014.30 |
| Infect/Incubation | Cytodex 3 microcarrier beads | 1,500 | g | $5.38 | $8,070.00 |
| | DMEM Media | 300,000 | mL | $0.04 | $12,450.00 |
| | Calf Serum(5%) | 15,000 | mL | $0.34 | $5,145.00 |
| | Overhead Hrs | 64 | Hrs | | $0.00 |
| | | | | Total | $25,665.00 |
| | 2 operators - 12-16 hours | | | | |
| Harvest | 5 L Biocontainer | 60 | Ea | $53.00 | $3,180.00 |
| | Sucrose | 30,000 | mL | $0.04 | $1,200.00 |
| | Overhead Hrs | 8 | Hrs | | $0.00 |
| | 2 operators - 4-8 hours depending on if diafiltered | | | Total | $4,380.00 |
| | and/or stabilized | | | | |

| | Item | Quantity Used | Unit | Cost/Unit | Total cost |
|---|---|---|---|---|---|
| Final Product | Pichinde Antigen (1.0 × 10^{8.0} PFU/mL) | 300,000 | mL | 0.12 | $37,059.30 |

In addition, the rP18tri-HEV-fiber-cFL construct having the gene sequence coding for the fiber protein (full length) was highly efficacious in reducing splenomegaly; as the concentration of PFU decreased from 1.0×10⁷ to 1.0×10⁴ the size of the spleen increased as shown in FIG. 7. The spleen/body weight ratio of the rP18tri-HEV-fiber-(cFL) was significantly lower (1.04) compared to the vector control (1.75) and Oralvax (1.59) respectively at a single dose of 1.0×10⁵ PFU (FIG. 7). These data show that the codon optimized rP18tri-HEV-fiber-cFL viral vector induced protective immunity against an HEV challenge in turkeys as seen by the decrease in splenomegaly.

### Example 13

The Efficacy of the rP18tri-HEV-Fiber-FL Protein Against Hemorrhagic Enteritis Virus in Turkeys when Given at Day of Age

The purpose of this experiment was to evaluate the protective efficacy of rP18tri-HEV-fiber-FL vector having the gene sequence coding for the Fiber protein (full length) given at 1-day of age compared to the vaccine given at three weeks of age against an HE challenge. The gene sequence was not codon optimized to avian species. The outcome parameters used to evaluate vaccine efficacy were 1) evaluate the efficacy of the rP18tri-HEV-fiber-FL vector when given at day of age, 2) compare the efficacy of the vector when given at day-1 compared to the vector given at three weeks of age (day-21) compared to the commercial vaccine (Oralvax) given at 3-weeks of age.

Briefly, 125-day old turkeys poults obtained from Select Genetics (Willmar, MN) were leg banded for identity and equally divided into 5 groups (25 birds per group) designated as Groups 1-5; Group-1 (placebo-non-vaccinated/non-challenged), Group-2 (Vector control-rP18tri-GFP), Group 3 (rP18tri-HEV-fiber-FL vector vaccinated at day-1 of age), Group-4 (rP18tri-HEV-fiber-FL vector vaccinated at three weeks of age) and Group-5 (Oralvax vaccinated at three weeks of age). At day-1 of age birds in Group 3 were vaccinated subcutaneously with the rP18tri-HEV-fiber-FL vector at a dose of 1.0×10⁷ PFU per 1.0 ml. Birds in Group-5 were separated into a separate isolation room and at the appropriate time were orally gavaged with the live Oralvax HE vaccine at the manufactures recommended dose on day-21. Twenty-one days after vaccination (day-42) birds in Group-5 were transported and co-mingled back into groups 1-5. All birds were then challenged by oral gavage with the virulent HEV at 1.0×10⁴ TCID₅₀ in a 1.0 ml volume. Table 8 shows the experimental design.

TABLE 8
Experimental design.

| | | | Vaccine | Vaccination/ | |
|---|---|---|---|---|---|
| Group | Turkey | Vaccine | Volume | Route | Challenge |
| 1 | 20 | Placebo non-vaccinated non- | None | None | None |
| | | challenged | | | |
| 2 | 20 | Vector control - rP18tri-GFP | None | IM (Day 21) | Day-42 |
| | | 10⁷ PFU | | | |
| 3 | 20 | rP18tri-HEV-fiber-(FL) 10⁷ | 1.0 ml | IM (Day 1) | Day-42 |
| | | PFU | | | |
| 4 | 20 | rP18tri-HEV-fiber-(FL) 10⁷ | 1.0 ml | IM (Day 21) | Day-42 |
| | | PFU | | | |
| 5 | 20 | Oralvax | 1.0 ml | Oral (Day 21) | Day-42 |

### Example 14

### Virulent HEV Challenge

At 42-days of age birds in Groups 2-5 were challenged by oral gavage with the virulent HEV at 1.0×10⁴ TCID₅₀ in a 1.0 ml volume. Birds in Group-1 remained as non-vaccinated/non-challenged. Morbidity and Mortality was monitored once daily for 7 consecutive days. At seven days post challenge, individual birds were weighted and then terminated. All birds were necropsied to evaluate vaccine efficacy based on the lack of splenomegaly and intestinal lesions of the duodenum.

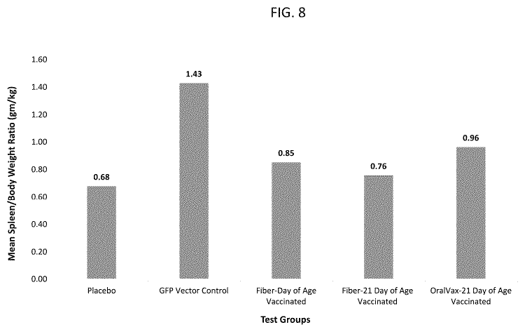

FIG. 8 Shows the comparative spleen/body weight ratio of vaccinates to control and Oralvax. The vector (rP18tri-HEV-fiber-FL) utilized the non-codon optimized sequence of the full-length fiber protein. This study evaluated the efficacy of vaccination using the rP18tri-HEV-fiber-FL given at day of age compared to vaccination given at three weeks of age (day-21) of both the rP18tri-HEV-fiber-FL and Oralvax. Vaccinated birds were challenged at six weeks of age (day-42). Please note: the protective efficacy after challenge of the vector rP18tri-HEV-fiber-(FL) given at day of age (day-1) with a spleen/body weight ratio of 0.85 compared to Oralvax given at three weeks of age (day 21) and the GFP vector control showing a Spleen/Body weight ratio of 0.96 and 1.43 respectively. In addition, there was no statistical difference in the efficacy of the vector given at day of age compared to the vaccine given at three weeks of age (day 21) with a Spleen/Body weight ratio of 0.85 and 0.76.

These results clearly demonstrate that the rP18tri-HEV-fiber-(FL) vector having the gene sequence coding for the Fiber protein (full length) of Hemorrhagic Enteritis Virus can be effectively administered to turkey poults at 1-day of age without the interference or neutralization of the live HE vaccine due to maternal antibody. This interference is a common problem in the turkey industry due to the passive transfer of HEV antibodies from the turkey breeder hen to its progeny via the egg. The results showed no difference in the spleen/body weight ratio of birds vaccinated at day of age compared to birds vaccinated at three weeks of age (FIG. 8).

### Example 15

Vector Clearance in Tissues of the Codon Optimized Vector rP18tri-HEV-Fiber-cFL in Turkeys

The purpose of this of experiment was to evaluate the clearance of the codon optimized vector rP18tri-HEV-fiber-cFL in tissues of vaccinated birds. Briefly, thirty-five day old turkey poults obtained from Select Genetics (Willmar, MN) were raised in isolation and vaccinated at 6 weeks of age with the rP18tri-HEV-fiber-cFL at 1.0×10⁷ PFU. Three birds were necropsied on each of the following days: 0 (day of vaccination), 1, 2, 3, 5 and 7. At necropsy, tissues were obtained from breast muscle (at the site of injection), blood (in heparin tubes), liver and spleen. Tissues were homogenized and tested for the presence of rP18tri PICV using a PCR assay.

A PCR assay was used to detect the presence of the vector in sampled tissues. The tissues were homogenized in a bead-based homogenizer using 0.5 ml of Phosphate Buffered Saline (PBS) pH 7.2. Following homogenization, the tubes were centrifuged at 10,000 RPM at 40C for 10 min and the supernatant was collected. Two hundred microliters (200 μl) of the supernatant was used for viral RNA extraction using the GeneJET Viral DNA and RNA Purification Kit #K0821 (Thermo scientific) and the viral RNA was extracted in a 20 μl elution volume. For the blood samples, 200 μl of blood was used for viral RNA extraction. Briefly, 5 μl of the Extracted RNA was subjected to one-step RT-PCR using verso enzyme mix (Thermo scientific) for detection of Pichinde virus RNA The Following PCR primers were used:

Primer Sequences:
Forward primer:
3′-ACC AGG GAA GAG TGC AG-5′
Reverse primer:
3′-AGG TGA ACA GCA TCA CAG ACT TG-5

The PCR reaction was performed in 50 μl volumes, containing 5 μl of template plasmid DNA, 5 μl of 10× buffer, 5 μl of 10 mM dNTPs, 1 μl of Forward primer (100 μM concentration), 1 μl of Reverse primer (100 μM concentration), 0.5 μl of Phusion Taq Polymerase and 32.5 μl of DNase/RNase free water. The PCR Cycling conditions were as follows: initial denaturation 5 minutes at 95° C. followed by 35 cycles 95° C.-30s, 50° C.-45s, 72° C.-1 min; final extension 72° C.-5 min. A PCR product of 1.3 kb corresponding to full length HEV Fiber gene was obtained. The PCR products were resolved on a 2% Agarose gel.

The results showed that the rP18tri-HEV-fiber-cFL vector causes a limited transient infection without inducing viremia. The recombinant viral vector upon administration is restricted to the lymphatic system and is cleared from the animal within 5 days of vaccination. A PCR assay using Pichinde NP gene specific primers with a limit of detection 10 RNA copies per reaction was used to detect the spread of rP18tri-HEV-fiber-cFL vector in tissues of vaccinated turkeys. The vector was only briefly detected in spleen before being cleared by Day 5 (Table 9) and was not detected in blood or any of the remaining tissues examined.

TABLE 9
Tissue tropism of the rP18tri-HEV-fiber-(cFL) vaccine in turkeys (vaccinated with 1.0 × 10⁷ PFU)

| | Day 1 | | | Day 3 | | | Day 4 | | | Day 6 | | | Day 7 | | | Day 8 | | |
|---|---|---|---|---|---|---|---|---|---|---|---|---|---|---|---|---|---|---|
| | Bird | Bird | Bird | Bird | Bird | Bird | Bird | Bird | Bird | Bird | Bird | Bird | Bird | Bird | Bird | Bird | Bird | Bird |
| Samples | 1 | 2 | 3 | 1 | 2 | 3 | 1 | 2 | 3 | 1 | 2 | 3 | 1 | 2 | 3 | 1 | 2 | 3 |
| Blood | ND | ND | ND | ND | ND | ND | ND | ND | ND | ND | ND | ND | ND | ND | ND | ND | ND | ND |
| Breast | +++ | +++ | +++ | ND | ND | ND | ND | ND | ND | ND | ND | ND | ND | ND | ND | ND | ND | ND |
| Muscle | | | | | | | | | | | | | | | | | | |
| Spleen | ND | ND | ND | +++ | +++ | +++ | +++ | +++ | +++ | ND | ND | ND | ND | ND | ND | ND | ND | ND |
| Liver | ND | ND | ND | ND | ND | ND | ND | ND | ND | ND | ND | ND | ND | ND | ND | ND | ND | ND |

ND—Not detected (limit of detection at 10 RNA copies per reaction (~0.2 Grams of spleen tissue)

The present disclosure is not to be limited in scope by the specific embodiments described herein. Indeed, various modifications of the disclosure in addition to those described herein will become apparent to those skilled in the art from the foregoing description. Such modifications are intended to fall within the scope of the appended claims. All publications referred to herein are hereby incorporated by reference.

1. A genetically engineered Pichinde virus comprising:
a first genomic segment comprising a coding region encoding a Z protein and a coding region encoding an L RNA-dependent RNA polymerase protein;
a second genomic segment comprising a coding region encoding a glycoprotein (GPC);
a third genomic segment comprising a coding region encoding a nucleoprotein (NP);
wherein at least one of the second and the third genomic segment further comprises a donor gene sequence, wherein the donor gene sequence encodes an adenovirus capsid protein or a fragment thereof.

2. The virus of claim 1, wherein the second genomic segment comprises a multiple cloning site (MCS) and the donor gene sequence, wherein the MCS comprises a first enzyme restriction site, and wherein the donor gene sequence is located at the first enzyme restriction site.

3. The virus of claim 1, wherein the third genomic segment comprises a MCS and the donor gene sequence, wherein the MCS comprises a second enzyme restriction site, and wherein the donor gene sequence is located at the second enzyme restriction site.

4. The virus of claim 1, wherein the adenovirus capsid protein is a full-length hemorrhagic enteritis virus (HEV) capsid protein.

5. The virus of claim 1, wherein the donor gene sequence comprises a nucleic acid sequence having at least 80% identity to HEV Fiber Protein.

6. The virus of claim 1, wherein each of the genomic segments further comprises a regulatory sequence selected from the group consisting of a promotor, a transcription initiation start site, a Kozak consensus sequence, a ribosome binding site, an RNA processing signal, a transcription termination site, a polyadenylation signal, or a combination thereof.

7. The virus of claim 1, wherein at least one of the genomic segments further comprises a reporter gene sequence encoding a detectable marker.

8. The virus of claim 1, further comprising a fourth genomic segment encoding a T7 RNA polymerase.

9. An infectious virus particle comprising the genomic segments of claim 1.

10. A composition comprising the isolated infectious virus particle of claim 9.

11-54. (canceled)
